(19) United States
(12) Patent Application Publication
(10) Pub. No.: US 2025/0277187 A1
(43) Pub. Date: Sep. 4, 2025

(54) A METHOD FOR PRODUCING INDUCED STEROIDOGENIC CELLS AND USE THEREOF IN CELL THERAPY

(71) Applicant: TECHNISCHE UNIVERSITAET DRESDEN, Dresden (DE)

(72) Inventors: Gerard RUIZ BABOT, Dresden (DE); Stefan R. BORNSTEIN, Dresden (DE)

(21) Appl. No.: 18/708,096

(22) PCT Filed: Nov. 9, 2022
(86) PCT No.: PCT/EP2022/081212

§ 371 Date: May 7, 2024

Related U.S. Application Data
(60) Provisional application No. 63/263,795, filed on Nov. 9, 2021.

(30) Foreign Application Priority Data
Mar. 7, 2022 (EP) 22160480.4

(51) Int. Cl. C12N 5/071; A61K 35/22

(52) U.S. Cl. CPC C12N 5/0614; A61K 35/22; C12N 2501/60; C12N 2506/45; C12N 2510/00

(57) ABSTRACT

The invention relates to a method for producing induced steroidogenic cells (iSCs) cells from an initial cell, the method comprising: providing at least one transcription factor (TF) to the initial cell, comprising at least steroidogenic factor 1 (SF-1), wherein in a preferred embodiment the initial cell is a pluripotent cell. The invention further relates to an induced steroidogenic cell (iSC), either generated according to the method of the invention or comprising an exogenous nucleic acid comprising an SF1-encoding region operably linked to a promoter or promoter/enhancer combination. The invention further relates to said iSC for use as a medicament, preferably for use in the treatment of either steroid deficiency, adrenal insufficiency or congenital adrenal hyperplasia. The invention further relates to a kit for producing said iSC, an expression vector and for a method for producing steroidogenic hormones in an iSC.

The invention relates to a method for producing induced steroidogenic cells (iSCs) from an initial cell, the method comprising: providing at least one transcription factor (TF) to the initial cell, comprising at least steroidogenic factor 1 (SF-1), wherein in a preferred embodiment the initial cell is a pluripotent cell. The invention further relates to an induced steroidogenic cell (iSC), either generated according to the method of the invention or comprising an exogenous nucleic acid comprising an SF1-encoding region operably linked to a promoter or promoter/enhancer combination. The invention also relates to the iSC for use as a medicament, preferably for use in the treatment of either steroid deficiency, adrenal insufficiency or congenital adrenal hyperplasia. The invention further relates to a kit for producing said iSC, an expression vector and to a method for producing steroidogenic hormones in an iSC.

### BACKGROUND OF THE INVENTION

Adrenal glands are essential for life and control fundamental physiological functions through steroid synthesis and hormone secretion. Located above the kidney, the adrenal plays a critical role in the regulation of volume status and salt homeostasis, carbohydrate metabolism and is a central mediator of the stress response. The adrenal cortex is an important steroid-producing organ that releases glucocorticoids under the control of adrenocorticotropic hormone (ACTH) secreted by the anterior pituitary gland, and mineralocorticoids under the control of the renin-angiotensin system. Primary or secondary adrenal insufficiency results from adrenal failure or impairment of the hypothalamic-pituitary axis. In either case, the adrenal cortex is unable to secrete sufficient amounts of glucocorticoids and adrenal androgens. Primary adrenal insufficiency, the inability to produce adrenal steroids is a life-threatening disorder that requires lifelong hormone replacement and can cause significant morbidity related to chronic fatigue, muscle weakness, loss of appetite and weight loss. To date, however, hormone replacement therapy serves to palliate these symptoms only partially and can lead to significant side effects in patients receiving inappropriate doses. Current treatments for steroid deficiencies in the adrenal field are based on hormone replacement therapy (HRT), consisting of 2-3 daily doses of glucocorticoid, and mineralocorticoid replacement in some cases. Indeed, over- or under-substitution with glucocorticoids lead to reduced vitality and perception of general health accompanied by reduced physical function resulting in reduced working capacity. Other complications include hypertension and several metabolic alterations.

Cortisol patterns in healthy individuals follows a circadian rhythm that is difficult to mimic with current HRT regimes, with periods of under and over replacement. Inappropriate levels of glucocorticoid can result in reduced vitality and perception of general health accompanied by diminished physical function. Chronic over-treatment with glucocorticoid therapy can also lead to hypertension and metabolic syndrome. Even when carefully treated, patients remain at risk for disease-associated complications when they become ill, injured or are under physical stress, requiring extra medication during these times. If untreated, hormone deficiencies can lead to adrenal crisis and death.

The use of cell therapy in the treatment of adrenal insufficiency has been attempted before by Sonoyama et al. 2012 and Matsuo et al. 2017. Both studies cultivate pluripotent cells in embryo-like aggregates. The disadvantages of cultivating pluripotent cells in embryo-like aggregates is that differentiation progresses slowly, while not every cell differentiates into the desired lineage. Hence, after differentiation the desired cells need to be isolated from the aggregates and separated from undesired cells by laborious cell sorting. Hence, high-throughput production of adrenal gland-derived hormones is not feasible with these methods. Many prior art methods, such as the afore mentioned, as well as Tanaka et al. 2020, Yazawa et al. 2016, US20080313752 and WO2019195828 use undefined culture media comprising serum in their approaches of generating hormone producing cells. As serum comprises a multitude of factors, such culture media offer less controlled differentiation conditions and hence reduce the output of cells showing the desired differentiation status. WO2019195828 describes methods for generating hormone producing cells of the gonad. No prior art method provides means enabling production of aldosterone, corticosterone and cortisol.

Others, such as Miyamoto et al., 2011 and WO 2006/038462 A1, used multipotent cells, such as mesenchymal stem cells or bone marrow cells, for the generation of steroid-producing cells through the infection with retro- or adenoviral expression-constructs encoding SF-1. However, steroidogenic cells generated from multipotent cells lack prolonged proliferation and expansion potential, such that culture-expansion and long-term maintenance of the generated steroidogenic cells is limited.

In summary, no prior art method provides means for controlled and high-throughput production of cells, capable of secreting all adrenal-gland derived hormones, which can be used for the cell therapy of adrenal-insufficiency and other related diseases.

In light of the prior art there remains a significant need in the art to provide new approaches for generating steroid-producing cells and generating means for treating adrenal insufficiency or other steroid deficiency-related diseases.

### SUMMARY OF THE INVENTION

In light of the prior art the technical problem underlying the present invention is to provide improved or alternative means for generating steroid-producing cells and/or for treating adrenal insufficiency and other steroid deficiencies.

This problem is solved by the features of the independent claims. Preferred embodiments of the present invention are provided by the dependent claims.

The inventors developed a new approach of providing human cells that could be used as a cell replacement platform for use in the treatment of patients with adrenal insufficiency and other steroid deficiencies.

The invention therefore relates to a method for producing induced steroidogenic cells (iSCs) cells from an initial cell, the method comprising: providing at least one transcription factor (TF) to the initial cell, comprising at least steroidogenic factor 1 (SF-1).

In one embodiment of the method according to the invention for producing induced steroidogenic cells (iSCs) from an initial cell, the initial cell is a pluripotent cell.

In another embodiment of the method according to the invention for producing induced steroidogenic cells (iSCs) cells from an initial cell, the initial cell is an induced pluripotent stem cell (iPSC).

In a further embodiment of the method according to the invention for producing induced steroidogenic cells (iSCs) cells from an initial cell, the initial cell is an pluripotent stem cell line, preferably an induced pluripotent stem cell line or a pluripotent stem cell line derived from iPSCs.

In a further embodiment of the method according to the invention for producing induced steroidogenic cells (iSCs) cells from an initial cell, the initial cell is an embryonic stem cell, preferably a human embryonic stem cell.

In a further embodiment of the method according to the invention for producing induced steroidogenic cells (iSCs) cells from an initial cell, the initial cell is an embryonic stem cell line, preferably a human embryonic stem cell line.

Any embodiment or example presented herein with an ESC, may be conducted in an analogous manner with a PSC, or iPSC. Embodiments or examples with iPSC may be conducted with ESC or PSC. Embodiments or examples with PSC may be conducted with ESC or iPSC. Any feature disclosed in any one or more of such embodiments or examples is considered disclosed with respect to the other cell types. Such embodiments may be adapted accordingly by a skilled person.

In some embodiments the steroidogenic cells is sensitive and/or responsive to ACTH (adrenocorticotropic hormone). In this embodiment the cells and hence the steroid production follow the circadian rhythm and are able to react with an appropriate hormone secretion depending on the time and dose.

The inventors developed a new method for generating steroid-producing cells from human pluripotent stem cells (hPSCs), providing a significant improvement over the methods known from the prior art. It was entirely surprising that the present method enables the generation of billions of cells that are able to produce steroid hormones from human pluripotent stem cells, both human embryonic stem cells (hESCs) and human induced pluripotent stem cells (iPSCs). To generate those cells, the present method uses a lentivirus that overexpress the transcription factor steroidogenic factor 1 (SF-1, gene NR5A1). Once the cells are infected with the lentivirus comprising the SF1-gene, they express steroidogenic enzymes de novo and secrete detectable levels of steroidogenic hormones in the medium. The surprising technical effect of the steroid producing cells generated according to the invention is that they can be used in the treatment of adrenal insufficiency. Until today no hESC or iPSC-based treatment is known and the currently used standard hormone replacement therapy that has many limitations and provides numerous disadvantages, such as side effects of inappropriate glucocorticoid substitution, inadequate diurnal rhythm of glucocorticoid levels and absent glucocorticoid release upon stress.

Compared to current treatments with hormone replacement therapy the use of steroid-producing cells according to the invention offers a clear clinical advantage in steroid hormone homeostasis and patient wellbeing, as the present invention facilitates the restoration of physiological hormone secretion, reconstitution of hypothalamic-pituitary-adrenal (HPA) endocrine axis and minimizes clinical symptoms and side effects of current treatment approaches.

The invention relates in one embodiment to a method for producing large quantities of steroid-producing cells from either (human) induced pluripotent stem cells or from human embryonic stem cells (or cell lines). In one embodiment the pluripotent stem cells are infected with a corresponding lentiviral vector so that the transcription factor SF1 is overexpressed, and steroid hormones are secreted. In some embodiments it may be crucial that the steroid-producing cells can also be regulated by the ACTH (adrenocorticotropic hormone), whereby they follow the circadian rhythm and can react with an appropriate hormone secretion depending on the time and dose. In contrast to conventional hormone replacement therapy, embodiments of the present invention have the advantage that the pulsating circadian release of steroid hormones, which are required during the day and night, is restored.

In embodiment of the present invention relating to the treatment of patients with adrenal insufficiency or other steroid deficiencies (cell therapy), immunoisolating devices are used to engulf or isolate, nourish and at the same time protect the cells that were generated according to the invention from a patient's immune system. The use of immunoisolating devices enables an easy monitoring of cells and enables the replacement of cells, avoiding the concerns about carcinogenesis of the cells or immune rejection. Immunoisolating devices can be partially or entirely implanted into the patient or be connected to the patient body by appropriate means. The implantation and/or connection of the device to the patient's body enables a constant exposure of the patient to the cells and their excreted products, such as steroid hormones.

Hence, in one embodiment the present invention also relates to the use of immunoisolating devices to protect the cells generated according to the present invention from the immune system of a (human) patient. This method already is applied in clinical trials for treatments targeting diabetes.

The general purpose of the present invention is the treatment of patients with adrenal insufficiency or other steroid deficiencies (i.e., hypogonadism). The overall significance and importance of this technology is the ability to transplant steroidogenic cells that 1) can be properly regulated by stimuli from the hypothalamus-pituitary-adrenal (HPA) axis and the renin-angiotensin-aldosterone system (RAAS) and 2) can respond with adequate hormone secretion in a time and dose dependent manner. Using this cellular approach, patients will have the additional potential advantage of restoring the pulsatile and circadian release of hormones needed throughout the day/night, one of the major challenges inherent to conventional hormone replacement therapies.

In one embodiment these steroidogenic cells generated according to the invention produce mineralocorticoids, and/or glucocorticoids, and/or androgens. In one embodiment these steroidogenic cells generated according to the invention also produce sex steroids, which could be used in the therapy of patients lacking gonadal hormones.

The invention is intended to enable in one embodiment the treatment of patients with adrenal insufficiency or other diseases which cause steroid deficiency, such as hypogonadism. If left untreated, adrenal insufficiency can lead to adrenal crisis and eventual death. Previous treatments have been based on hormone replacement therapies that are designed to mimic the circadian rhythm. However, this responsiveness to physiological needs remains limited in hormone replacement therapy. On the one hand, depending on the daily rhythm, there is a 10 phase-wise over- or undersupply of the corresponding steroid hormones. On the other hand, stopping hormone replacement therapy is very difficult, especially under stress and illness. As a result, patients show, for example, a severe impairment of well-being and a worsened general state of health (e.g., obesity, osteoporosis, high blood pressure, impaired glucose tolerance). Therefore, better treatment solutions are urgently needed. The present invention offers such an improved treatment solution. In contrast to conventional hormone replacement therapy, the cell therapy according to embodiments of the invention has the advantage that the pulsating circadian release of steroid hormones, which are required during the day and night, is restored.

In a preferred embodiment the invention relates to a method for producing induced steroidogenic cells (iSCs) cells from an initial cell, wherein the at least one TF is expressed from one or more exogenous nucleic acid molecules within the initial cell, preferably from one or more viral vectors, preferably lentiviral vectors. In other embodiments retroviral, adenoviral or adeno-associated viral vectors may be used.

In another embodiment the invention relates to a method for producing induced steroidogenic cells (iSCs) cells from an initial cell, wherein the initial cell is provided with the at least one TF for at least 3 days, preferably for at least 7. In other embodiments the initial cell is provided with the at least one TF for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 100, 150, 200, 250, 300, 350 or 365 days. In specific embodiments the initial cell is provided with the at least one TF permanently. In other embodiments the initial cell is provided with the at least one TF for between 1-365, 1-250, 1-100, 1-50, 5-25, 1-7, 1-14, 1-8, 7-14, 1-21, 1-28, 1-35, 1-42, 1-49 days or even between 1 day and several years in cases of long-term maintenance of a culture of cells according to the invention. In other embodiments the initial cell is provided with the at least one TF for between 1-2, 1-3, 1-4, 1-5, 1-6, 1-7, 1-8, 1-9, or 1-10 weeks or for between 1-2, 1-3, 1-4, 1-5, 1-6, 1-7, 1-8, 1-9, 1-10, 1-20, 1-30, 1-40, 1-50, 1-60, 1-70, 1-80, 1-90 or even 1-100 years. In another embodiment the initial cell is provided with the at least one TF at day 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 150, 200, 250, 300, 350 or day 365 of cultivation or after seeding/plating/thawing or passaging of the initial cell or at an even later time point.

In another embodiment the invention relates to a method for producing induced steroidogenic cells (iSCs) cells from an initial cell, wherein the at least one TF is expressed transiently and/or expression is induced in the initial cell.

In specific embodiments the expression of the at least one TF is inducible. In some embodiments the expression of the at least one TF is induced (initiated or started) by a certain stimulus, wherein the stimulus may in some embodiments be a certain substance, such as an antibiotic or a signaling molecule. In specific embodiments the antibiotic is selected from the group comprising blasticidin, doxycyclineromycin or tetracycline. In some embodiments the expression is induced at day 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 150, 200, 250, 300, 350 or day 365 of cultivation of the initial cell or at the respective day after providing at least one TF to the initial cell.

In specific embodiments the present method also relates to methods for the generation of steroid-producing 3-D cultures, 3-D cell aggregates or organoids from PSCs (pluripotent stem cells). According to some specific embodiments of the invention differentiation of PSCs, iPSC or (h) ESCs into steroidogenic cells can be achieved by recapitulating the well-established stages of human adrenocortical development using, preferably small molecules and morphogens, such as in one embodiment the transcription factor SF1. The morphogens may in one embodiment be introduced via an exogenic expression vector, e.g. using viral vectors.

In a further embodiment the present invention relates to steroid-producing organoids. In some embodiments said steroid-producing organoids are characterized by specific steroidogenic enzyme expression and/or hormone production.

In some embodiments the validation of these cells can be achieved by CRISPR-Cas9 gene editing of PSCs to engineer cell lines harboring mutations causing congenital adrenal hyperplasia (CAH)—the most frequent cause of primary adrenal insufficiency. In some embodiments differentiation of these cells will allow in vitro modelling of CAH. Some embodiments might also provide a proof-of-principle that gene editing can be used to modify enzymatic activity in steroid-producing organoids.

The present invention relates in another aspect to functional analysis of steroid-producing organoids using transplantation in animal models, e.g. mouse or rat models, of adrenal insufficiency. In some embodiments established murine models of adrenal insufficiency may be used to assess the potential of human steroid-producing organoids for cellular therapy. In further embodiments growth, survival and hormone production of organoids can be assessed, in vivo.

In some embodiments of the present invention the initial cell is a pluripotent stem cell line. In some embodiments the initial cell is an iPSC-derived pluripotent cell line.

In some embodiments of the present invention the initial cell is a human embryonal stem cell line. In some embodiments the initial cell is the H9 or H1 hESC line.

A stem cell line provides in some embodiments the following advantages i) Steroidogenic potential: The H9 hESC line can be reprogrammed toward the steroidogenic lineage using overexpression of SF16 (which can be used as a positive control); ii) Well established lines: The H9 hESC line has been fully characterized and is widely employed worldwide; iii) Excellent platform for disease modelling: CRISPR/Cas9-edited hESCs and isotype control lines represent an excellent model to assess the effect of a given mutation, avoiding comparisons between cells with a different genetic background. Moreover, CRISPR-Cas9 genome editing has been successfully achieved before for multiple loci in H9 hESC; iv) Reduced variability of experimental results: hESC provide a stable genetic background (compared to individual patient-derived iPSCs) and achieve reproducible differentiation outcome v) Pluripotency and self-renewal capacity: hESCs are able to generate any cell type in the body and their self-renewal capacity may enable long-term maintenance of a stem cell pool within the organoid; In some embodiments the use of the H9 hESC line can provide a universal and homogeneous cell source to generate steroid-producing organoids with similar properties, which can be quality-controlled and in some embodiments encapsulated into immunoisolating chambers for future cellular therapy for patients suffering from adrenal insufficiency.

In some embodiments of the present methods for the generation of steroid-producing organoids from hESCs (human embryonal stem cells) the H9 hESCs cell line is used as initial cell to generate intermediate mesoderm (see for one embodiment FIG. 11). In some embodiments gene expression profiling can be performed by RT-qPCR and immunostaining to validate progression through each developmental stage (pluripotency markers: OCT4, SOX2; late primitive streak: TBXT; intermediate mesoderm markers: WT1, OSR1, HOXD11). In some embodiments, intermediate mesoderm generated from hESCs can be used as a screening platform to identify small molecules and/or morphogens with the capacity to induce SF1 expression. In some embodiments such factors include those known to be involved in adrenocortical development or known to modulate adrenal signaling pathways (Wnt4, Rspo1, Rspo3, CHIR99021, BIO, SANT1, cyclopamine, Foskolin, db-CAMP, br-CAMP FGF-2, FGF-9, Activin-A, inhibin-a, SB431542, IGF-1, IGF-2, Dasatinib, pazopanib, Rottlerin, retinoic acid and BMP4). In some embodiments the over-expression of SF1 in intermediate mesoderm cells, using viral transduction, may be used as a positive control for induction into the steroidogenic lineage.

In some embodiments the overexpression of ACTH receptor (MC2R) and/or its co-receptor (MRAP) may be used to increase the basal and ACTH-stimulated cortisol production in iSCs. In some embodiments bi-cistronic vectors encoding MC2R and/or MRAP under a specific promoter, preferably the CMV promoter, may be used to generate lentiviral vectors and/or lentivirus particles according to the invention (See FIG. 14B and Table 1, column 1).

In embodiments of the method for producing induced steroidogenic cells (iSCs) from an initial cell, the method further comprises providing an ACTH receptor and/or its co-receptor to the initial cell. In embodiments the ACTH receptor is MC2R and its co-receptor is MRAP.

In embodiments of the method for producing induced steroidogenic cells (iSCs) from an initial cell, the method comprises expression of at least one transcription factor (TF) and/or an ACTH receptor and/or its co-receptor.

In embodiments of the method for producing induced steroidogenic cells (iSCs) from an initial cell, the method comprises expression of at least one transcription factor (TF), comprising at least steroidogenic factor 1 (SF-1), and/or the ACTH receptor MC2R and/or its co-receptor MRAP.

In embodiments said gene(s) are expressed in the initial cell. In embodiments said gene(s) are expressed in the iSCs. In embodiments said gene(s) are provided to the initial cell and expression is induced at a specific time or said gene(s) are expressed continuously.

In embodiments of the method for producing induced steroidogenic cells (iSCs) from an initial cell, the method comprises overexpression of at least one transcription factor (TF) and/or an ACTH receptor and/or its co-receptor.

In embodiments of the method for producing induced steroidogenic cells (iSCs) from an initial cell, the method comprises overexpression of at least one transcription factor (TF), comprising at least steroidogenic factor 1 (SF-1), and/or the ACTH receptor MC2R and/or its co-receptor MRAP.

In embodiments the at least one transcription factor (TF) and/or ACTH receptor and/or its co-receptor are expressed from one or more exogenous nucleic acid molecules within the initial cell and/or the iSCs.

In embodiments the method comprises providing to the initial cell at least one transcription factor (TF), an ACTH receptor and its co-receptor. In embodiments the method comprises expression of at least one transcription factor (TF), an ACTH receptor and its co-receptor. In embodiments the ACTH receptor is MC2R, the co-receptor is MRAP and the at least one transcription factor (TF) comprises at least steroidogenic factor 1 (SF-1).

In embodiments the one or more exogenous nucleic acid molecules are viral vectors, preferably lentiviral vectors.

In embodiments the method comprises infecting the initial cell with one or more viral particles, preferably lentiviral particles, comprising one or more exogenous nucleic acid molecules.

In embodiments the one or more exogenous nucleic acid molecules comprise an SF1-encoding region and/or a region encoding MC2R and/or a region encoding its co-receptor MRAP.

In embodiments of the method for producing induced steroidogenic cells (iSCs) from an initial cell, the iSCs exhibit an enhanced ACTH responsiveness, preferably compared to cells without or with lower ACTH receptor and/or co-receptor expression. In embodiments the enhanced ACTH responsiveness is at least partially due to the (over) expression of the ACTH receptor and/or its co-receptor.

In some embodiments of the method for producing induced steroidogenic cells (iSCs) from an initial cell, the method comprises:
providing at least one transcription factor (TF) to the initial cell, comprising at least steroidogenic factor 1 (SF-1), and additionally providing the ACTH receptor MC2R and/or its co-receptor MRAP to the initial cell, wherein preferably the at least one TF and/or MC2R and/or MRAP are expressed from one or more exogenous nucleic acid molecules within the initial cell, preferably from one or more viral vectors, preferably lentiviral vectors. In embodiments SF-1 and both MC2R and MRAP are expressed from one or more exogenous nucleic acid molecules. In preferred embodiments MC2R and MRAP are expressed bicistronically from the same exogenous nucleic acid molecule.

In embodiments the method for producing induced steroidogenic cells (iSCs) from an initial cell comprises providing at least one transcription factor (TF) to the initial cell, comprising at least steroidogenic factor 1 (SF-1), and additionally providing an ACTH receptor, preferably MC2R, to the initial cell. In embodiments the ACTH receptor, preferably MC2R, is expressed from an exogenous nucleic acid molecule within the initial cell, preferably from a viral vector, preferably a lentiviral vector.

In some embodiments the method for producing induced steroidogenic cells (iSCs) from an initial cell comprises providing at least one transcription factor (TF) to the initial cell, comprising at least steroidogenic factor 1 (SF-1), and additionally providing an ACTH-receptor co-receptor, preferably MRAP, to the initial cell. In embodiments the ACTH-receptor co-receptor, preferably MRAP, is expressed from an exogenous nucleic acid molecule within the initial cell, preferably from a viral vector, preferably a lentiviral vector.

In some of said embodiments MC2R and MRAP are expressed from the same exogenous nucleic acid molecule within the initial cell, preferably from the same viral vector, more preferably from the same lentiviral vector (see e.g., FIGS. 7B and 14B). In other embodiments MC2R and MRAP are expressed from two separate exogenous nucleic acid molecules within the initial cell, preferably from two viral vectors, more preferably from two lentiviral vectors.

As shown in FIG. 7E, upon the presence of the ACTH receptor MC2R and/or its co-receptor MRAP in a cell, ACTH stimulation induces an intracellular signaling cascade leading to the upregulation of production and secretion of cortisol into the culture supernatant. Experimental proof of the increased ACTH-responsiveness of the cells according to the invention, upon infection with a lentiviral vector encoding MC2R and MRAP is shown in the bar graph at FIG. 15B-D.

Without being bound by theory, it is observed surprisingly that the unexpected synergistic effect of the exogenous expression of SF-1 and MC2R and/or its co-receptor MRAP of improved responsiveness of the present cells to ACTH may be at least partially due to insufficient expression of the ACTH downstream machinery upon sole expression of exogenous SF-1. When only SF-1 is expressed exogenously a functional ACTH-downstream signaling appears to be lacking. Upon the exogenous expression of SF-1 and MC2R and/or its co-receptor MRAP the cells possess a functional ACTH responsiveness after the differentiation process.

In embodiments the exogenous nucleic acid molecules, preferably viral vectors, more preferably lentiviral vectors comprise a gene encoding a fluorescent protein. In some embodiments the fluorescent protein is expressed at the same time and only if the target gene(s), e.g., SF1, MRAP and/or MC2R, are expressed from said exogenous nucleic acid molecule. In some embodiments the target genes are expressed such that the protein encoded by said target genes comprises (is labelled with) the fluorescent protein. In preferred embodiments the target genes are expressed as separate protein(s) from the fluorescent protein, such that the target gene(s) are expressed as separate protein(s) (not fused with the fluorescent protein) and the fluorescent protein is expressed as separate protein. In said embodiments both/all genes are preferably expressed at the same time or nearly at the same time, such that the successful provision of the exogenous nucleic acid molecule encoding the target gene(s) to the cell can be detected by expression of the fluorescent protein. An example is disclosed in FIG. 16 (SF-1 is expressed together with the fluorescent protein EGFP) and a non-limiting example of a lentiviral expression vector backbone is disclosed in FIG. 18. In the experiment depicted in FIG. 16, the exogenous nucleic acid (viral expression vector shown in FIG. 18) encodes EGFP bicistronically with an IRES and SF-1, such that EGFP and SF-1 are expressed in the same cell and at the same time, but the “target gene” SF-1 itself is not labelled (no an EGFP-fusion gene).

Herein fluorescent proteins comprise, without being limited to, GFP, EGFP, YFP, BFP, mCherry, iRFP, and Azurite. The person skilled in the art knows suitable fluorescent proteins that can be selected and combined with target genes depending on the respective experimental context.

In another aspect the present invention relates to an induced steroidogenic cell (iSC) generated by the method according to the invention.

In another aspect the present invention relates to an induced steroidogenic cell (iSC) comprising an exogenous nucleic acid comprising an SF1-encoding region operably linked to a promoter or promoter/enhancer combination, wherein the induced steroidogenic cell (iSC) is preferably genetically modified with said exogenous nucleic acid.

In another embodiment the invention relates to a induced steroidogenic cell (iSC) comprising an exogenous nucleic acid comprising an SF1-encoding region operably linked to a promoter or promoter/enhancer combination, wherein the exogenous nucleic acid comprises a viral vector, more preferably a lentiviral vector.

In another embodiment the invention relates to an induced steroidogenic cell (iSC) comprising an exogenous nucleic acid comprising an SF1-encoding region operably linked to a promoter or promoter/enhancer combination, wherein the promoter is a constitutive or an inducible promoter.

In another embodiment the invention relates to an induced steroidogenic cell (iSC) comprising an exogenous nucleic acid comprising an SF1-encoding region operably linked to a promoter or promoter/enhancer combination, wherein the induced steroidogenic cell (iSC) expresses SF-1 at levels above those expressed in an induced pluripotent stem cell (iPSC).

In another embodiment the invention relates to an induced steroidogenic cell (iSC) comprising an exogenous nucleic acid comprising an SF1-encoding region operably linked to a promoter or promoter/enhancer combination, wherein the cell comprises one or more of the features selected from the group comprising de novo expression of steroidogenic enzymes, de novo production of steroid hormones, adrenal stimuli responsiveness, in vitro growth and in vivo growth.

In embodiments the present induced steroidogenic cell (iSC) expresses the transcription factor SF-1.

In embodiments the present induced steroidogenic cell (iSC) overexpresses the transcription factor SF-1.

In embodiments the present induced steroidogenic cell (iSC) expresses an ACTH receptor and/or an ACTH receptor co-receptor.

In embodiments the present induced steroidogenic cell (iSC) overexpresses an ACTH receptor and/or an ACTH receptor co-receptor.

In embodiments the ACTH receptor is MC2R and the co-receptor is MRAP.

In embodiments the induced steroidogenic cell (iSC) expressing or overexpressing the ACTH receptor and/or ACTH receptor co-receptor exhibits an enhanced ACTH responsiveness, preferably compared to cells without or with lower ACTH receptor and/or co-receptor expression.

In some embodiments the invention relates to an induced steroidogenic cell (iSC) comprising one or more exogenous nucleic acid molecules comprising an SF1-encoding region and/or a region encoding MC2R and/or a region encoding its co-receptor MRAP. In embodiments the one or more exogenous nucleic acid molecules comprise regions encoding the ACTH receptor MC2R and its co-receptor MRAP bicistronically.

In some embodiments the invention relates to an induced steroidogenic cell (iSC) comprising one or more exogenous nucleic acid molecules comprising an SF1-encoding region and/or a region encoding MC2R and/or a region encoding its co-receptor MRAP, wherein said regions are operably linked to the same or separate promoter or promoter/enhancer combination(s).

In some embodiments the invention relates to an induced steroidogenic cell (iSC) comprising one exogenous nucleic acid molecule comprising an SF1-encoding region and one exogenous nucleic acid molecule comprising a region encoding MC2R and/or a region encoding its co-receptor MRAP, wherein said regions on each exogenous nucleic acid molecule are operably linked to the same or separate promoter or promoter/enhancer combination(s).

In embodiments the promoter of the promoter or promoter/enhancer combination(s) is a constitutive or an inducible promoter. In embodiments the promoter is a CMV promoter. In embodiments the enhances is a CMV enhancer.

In embodiments the exogenous nucleic acid molecules, preferably viral vectors, comprise and co-express MC2R and/or its co-receptor MRAP, preferably bicistronically.

In some embodiments an exogenous nucleic acid molecule comprises a viral vector, more preferably a lentiviral vector.

In embodiments the iSC according to the invention comprise one exogenous nucleic acid comprising an SF1-encoding region and one exogenous nucleic acid comprising a region encoding MC2R and/or a region encoding its co-receptor MRAP. In preferred embodiments of said exogenous nucleic acids the regions encoding MC2R and/or MRAP are operably linked to the same or separate promoter or promoter/enhancer combination(s).

In embodiments the iSC according to the invention comprise one exogenous nucleic acid comprising an SF1-encoding region and one exogenous nucleic acid comprising a region encoding MC2R and/or a region encoding its co-receptor MRAP, wherein the cell comprises one or more of the features selected from the group comprising de novo expression of steroidogenic enzymes, de novo production of steroid hormones, adrenal stimuli responsiveness, in vitro growth and in vivo growth.

In some embodiments the viral vectors may also comprise and co-express GFP or any other fluorescent protein, preferably bicistronically, and/or contain a mammalian resistance cassette, which can be used for selection. In some embodiments the expression of the steroidogenic acute regulatory protein (STAR) can be used as a readout for initial experiments, as transduction of SF1, can induce the expression of STAR.

In another aspect the present invention relates to an induced steroidogenic cell (iSC) for use as a medicament.

In further embodiments the present invention relates to an induced steroidogenic cell (iSC) for use in the treatment of a steroid deficiency.

In further embodiments the present invention relates to an induced steroidogenic cell (iSC) for use in the treatment of adrenal insufficiency.

In further embodiments the present invention relates to an induced steroidogenic cell (iSC) for use in the treatment of congenital adrenal hyperplasia (CAH).

In some embodiments the present invention relates to an induced steroidogenic cell (iSC) for use as a medicament, wherein the iSC comprises an exogenous nucleic acid comprising an SF1-encoding region operably linked to a promoter or promoter/enhancer combination, wherein the induced steroidogenic cell (iSC) is preferably genetically modified with said exogenous nucleic acid.

In some embodiments the present invention relates to an induced steroidogenic cell (iSC) for use as a medicament, wherein a plurality of iSCs, are encapsulated within an immunoisolating device. In further embodiments the present invention relates to an induced steroidogenic cell (iSC) for use in the treatment of a steroid deficiency, adrenal insufficiency or CAH (congenital adrenal hyperplasia), wherein a plurality of iSCs are encapsulated within an immunoisolating device. In embodiments the pluralities of iSCs comprise iSCs according to the present invention.

In another aspect the invention further relates to a method for producing steroidogenic hormones in an induced steroidogenic cell (iSC), the method comprising:
providing the iSC according to the present invention,
cultivating the iSC,
isolating hormones secreted by the cultivated iSC from the cell culture.

In some specific embodiments the initial cell is an iPSC that is derived from an individual patient, who is treated subsequently with cells or cell products according to the invention derived from his “own” iPSCs. This personalized medicine approach can in some embodiments reduce the attacks of the patients' immune system towards the introduced steroidogenic cells or cell products.

The present invention relates in a further aspect to a kit for producing induced steroidogenic cells (iSCs) from an initial cell according to the method of the present invention, comprising
a vector system for providing SF1/NR5A1, and optionally further proteins, to the initial cell, and
reagents for detecting induced steroidogenic cells generated from an initial cell, such as
   i. an antibody for the detection of one or more marker proteins, such as SF1, STAR, CYP11A1, CYP11A2, CYP11B1, CYP11B2, HSD3B2, HSD17B, HSD11B1, HSD11B2, CYP21A2, CYP17A1, GATA4, GATA6, Pax8, WT1, Dax1, MC2R, MRAP and/or Sox2, and/or
   ii. primers for detection of one or markers, such as NR5A1/SF1, STAR, CYP11A1, CYP11A2, HSD17B, HSD3B2, HSD11B1, HSD11B2, CYP21A2, CYP17A1, CYP11B1, GATA4, GATA6, Pax8, WT1, Dax1, Sox2, MC2R, MRAP or CYP11B2 by PCR.

In another aspect the present invention relates to an expression vector system comprising one or more nucleic acid sequences operably coupled to one or more promoters, wherein said sequences encode one or more transcription factors (TFs) comprising at least SF1 and optionally further proteins.

In one embodiment the invention relates to a method for differentiating hESCs into steroidogenic cells. This can in some embodiments be achieved through optimization of the protocols for generating steroid-producing organoids or 3-D cultures from hESCs, recapitulating the initial stages of human adrenocortical development, using small molecules and steroidogenic factor 1 (SF-1) overexpression. This specific embodiment aims to employ a transgene-free strategy to induce SF-1 ex-pression to generate functional and hormonally responsive steroidogenic organoids.

In one embodiment the present invention relates to method of treating CAH patients, using hormone-producing, stimuli-responsive steroidogenic cells (iSCs) that could overcome some of the present treatment limitations. The overall significance and importance of this embodiment in the field is the ability to transplant steroidogenic cells that 1) can be properly regulated by stimuli from the hypothalamus-pituitary-adrenal (HPA) axis and the renin-angiotensin-aldosterone system (RAAS) and 2) can respond with adequate hormone secretion in a time and dose dependent manner. Using the cellular approach of this specific embodiment, patients can have the additional potential advantage of restoring the pulsatile and circadian release of hormones needed throughout the day/night, one of the major challenges inherent to conventional HRT.

The embodiments described herein for one aspect of the invention may also be embodiments of any one of the other aspects of the present invention. Accordingly, embodiments described for the method according to the invention may also be embodiments of the iSC cells, the use of the cells as medicament, the method for producing steroidogenic hormones and the kit disclosed herein. In addition, any embodiment described herein, may also comprise features of any other embodiment of the invention.

### DETAILED DESCRIPTION OF THE INVENTION

All cited documents of the patent and non-patent literature are hereby incorporated by reference in their entirety.

In one embodiment the present invention relates to a method for producing induced steroidogenic cells (iSCs) cells from an initial cell, the method comprising: providing at least one transcription factor (TF) to the initial cell, wherein the at least one transcription factor (TF) is SF1, wherein the initial cell may in some embodiments be a pluripotent cell that is preferably either an embryonal stem cell, an embryonic stem cell line, an induced pluripotent stem cell (iPSC), an induced pluripotent stem cell line or a pluripotent cell line derived from iPSCs.

Key hormones produced by the adrenal gland include: Cortisol, Aldosterone, DHEA and Androgenic Steroids, Epinephrine (Adrenaline) and Norepinephrine (Noradrenaline). The adrenal cortex produces three hormones mineralocorticoids (the most important is aldosterone), glucocorticoids (predominantly cortisol) and adrenal gland androgens, such as male sex hormones mainly dehydroepiandrosterone (DHEA) and testosterone. Adrenocorticotropic hormone (ACTH), secreted by the anterior pituitary gland, primarily affects release of glucocorticoids and adrenal androgens by the adrenal gland and, to a much lesser extent, also stimulates aldosterone release. The adrenal medulla produces catecholamine including adrenaline, noradrenaline and small amounts of dopamine.

The ‘adrenocorticotropic hormone receptor’ (ACTH receptor), further known as melanocortin receptor 2 (MC2R), is a type of melanocortin receptor (type 2). MC2R is specific for ACTH and is understood to require the binding to melanocortin-2 receptor accessory protein-1 (MRAP1) to bind ACTH.

Herein, CAH refers to congenital adrenal hyperplasia (CAH), caused by mutations in steroidogenic enzymes, is the most frequent cause of primary adrenal insufficiency with a prevalence of about 1 in 18,000 worldwide. Defects in the CYP21A2 gene is the most common form of CAH.

The method of the invention relates to providing one or more transcription factors. Providing a transcription factor or other factor, such as a micro-RNA, in the context of the present invention relates to provision or making available or contacting a TF with the initial cell or introducing the TF within the cell, or having the TF produced from within or in close proximity to the initial cell. The TF may be provided at the protein level or in the form of a nucleic acid encoding a TF. Accordingly, in case of delivery of an exogenous nucleic acid molecule encoding the TF, the TF is provided upon expression of the protein from the exogenous nucleic acid molecule. A TF can be provided through expression from any given nucleic acid molecule. This includes activation of expression of the respective TF from an endogenous or an exogenous nucleic acid molecule.

Furthermore, the TF can be delivered to the cell directly, for example by protein transfection. Preferably, the expression of a TF occurs in amounts greater than the initial cell, e.g. iPSCs.

TF provision can occur by expression from a nucleic acid molecule, such as an exogenous nucleic acid molecule. As used herein “nucleic acid” shall mean any nucleic acid molecule, including, without limitation, DNA, RNA and hybrids or modified variants thereof. An “exogenous nucleic acid” or “exogenous genetic element” relates to any nucleic acid introduced into the cell, which is not a component of the cells “original” or “natural” genome. Herein the terms “(exogenous) nucleic acid molecule” and “(exogenous) nucleic acid” may be used interchangeably. Exogenous nucleic acids may be integrated or non-integrated in the genetic material of the target stem cell or relate to stably transduced nucleic acids. Delivery of an exogenous nucleic acid may lead to genetic modification of the initial cell through permanent integration of the exogenous nucleic acid molecule in the initial cell. However, delivery of the exogenous nucleic acid may also be transient, meaning that the delivered genetic material for provision of the one or more TF disappears form the cell after a certain time.

Nucleic acid molecule delivery and potentially genetic modification of an initial cell, such as a mammalian or human cell, preferably a human iPSC, can be performed and determined by a skilled person using commonly available techniques. For example, for detecting genetic modification sequencing of the genome or parts thereof of an initial cell is possible, thereby identifying if exogenous nucleic acids are present. Alternatively, other molecular biological techniques may be applied, such as the polymerase chain reaction (PCR), to identify/amplify exogenous genetic material. Exogenous nucleic acids may be detected by vector sequences, or parts of vector sequences remaining at the site of genetic modification. In cases where vector sequences (for example vector sequences flanking a therapeutic transgene) can be removed from the genome, the addition of a therapeutic transgene may still be detected by sequencing efforts by detecting genomic sequences incorporating a therapeutic gene at a “non-natural” position in the genome.

Any given gene delivery method for delivery of nucleic acid molecules is encompassed by the invention and preferably relates to viral or non-viral vectors, as well as biological or chemical methods of transfection. The methods can yield either stable or transient gene expression in the system used. Furthermore, any method known to the person skilled in the art for delivery of proteins to a mammalian cell is encompassed by the present invention when referring to provision of one or more transcription factors and/or micro-RNAs or other factors. All known methods for delivery of nucleic acid molecules and proteins as well as other biological and chemical molecules that can act as factors in the context of the method of the invention are encompassed. This includes in particular microinjection, transfection, transduction, vesicle fusion and electroporation.

In one embodiment the invention relates to a method for producing an induced steroidogenic cell (iSC), wherein the TF is expressed from one or more exogenous nucleic acid molecules within the initial cell, wherein the exogenous nucleic acid comprises a viral vector, more preferably a lentiviral vector. Genetically modified viruses have been widely applied for the delivery of genes into mammalian cells and in particular stem cells. A viral vector may be employed in the context of the present invention.

Preferred viral vectors for genetic modification of the initial cells described herein relate to lentiviral vectors. A skilled person is aware of the techniques required for utilization of lentiviruses in genetic modification of pluripotent cells.

Lentiviruses are members of retroviridae family of viruses and lentivirus vectors are generated by deletion of the entire viral sequence with the exception of the LTRs and cis acting packaging signals. The resultant vectors have a cloning capacity of about 8 kb. One distinguishing feature of these vectors from retroviral vectors is their ability to transduce dividing and non-dividing cells as well as terminally differentiated cells.

The invention encompasses further the administration of expression vectors to a subject in need thereof. A “vector” is any means for the transfer of a nucleic acid into a host cell. A preferred vector relates to a replicon to which another DNA segment may be attached so as to bring about the replication of the attached segment. The term “vector” as used herein specifically refers to means for introducing the nucleic acid into a cell in vitro, ex vivo or in vivo. Viral vectors include, without limitation, retrovirus, adeno-associated virus, pox, baculovirus, vaccinia, herpes simplex, Epstein-Barr and adenovirus vectors.

Adenoviruses may be applied, or RNA viruses such as Lentiviruses, or other retroviruses. Adenoviruses have been used to generate a series of vectors for gene transfer cellular engineering. The initial generation of adenovirus vectors were produced by deleting the EI gene (required for viral replication) generating a vector with a 4 kb cloning capacity. An additional deletion of E3 (responsible for host immune response) allowed an 8 kb cloning capacity. Further generations have been produced encompassing E2 and/or E4 deletions.

An infection supernatant for viral infection of initial cells may comprise in some embodiments viral particles at a concentration (genome copies per mL) of, for example, 1×10³gc/ml, between 1-5×10³gc/ml, 1×10⁵gc/ml, between 1-5×10⁵gc/ml, 1×10¹⁰gc/ml, between 1-5×10¹⁰gc/ml, 1×10¹¹gc/ml, between 1-5×10¹¹gc/ml, 1×10¹²gc/ml, between 1-5×10¹²gc/ml, 1×10¹³gc/ml, between 1-5×10¹³gc/ml, 1×10¹⁴gc/ml, or between 1-5×10¹⁴gc/ml.

Non-viral methods may also be employed, such as alternative strategies that include conventional plasmid transfer and the application of targeted gene integration through the use of integrase or transposase technologies. These represent approaches for vector transformation that have the advantage of being both efficient, and often site-specific in their integration. Physical methods to introduce vectors into cells are known to a skilled person. One example relates to electroporation, which relies on the use of brief, high voltage electric pulses which create transient pores in the membrane by overcoming its capacitance. One advantage of this method is that it can be utilized for both stable and transient gene expression in most cell types. Alternative methods relate to the use of liposomes or protein transduction domains. Appropriate methods are known to a skilled person and are not intended as limiting embodiments of the present invention. Furthermore, delivery of RNA molecules such as mRNA transfection is included in the context of the method of the invention for provision of a TF from an exogenous nucleic acid.

Furthermore, delivery of exogenous nucleic acid molecules for provision of a factor may be achieved by means of a transposable element.

Provision of the TFs and other factors used in the method of the invention may be transient or permanent. For example, if provision is achieved by expression from a nucleic acid molecule, TF expression may be permanently active under the control of a constitutive promoter or a promoter that is active in the initial cell as well as in induced cells. Alternatively, expression and therefore provision of the TF may be transient, either because the nucleic acid molecule that encodes the TF is removed or disappears from the cell or because expression is controllable and can be turned on and off, for example by using controlled transcriptional activation. In the context of the present invention, transient expression refers to only temporal expression of a factor from a nucleic acid molecule in contrast to permanent expression.

Transient expression can in other examples occur through induction of gene expression from an exogenous DNA molecule comprising controllable genetic elements driving expression of the encoded gene, and therefore comprises inducible gene expression. In such systems gene expression can be externally controlled, for example through administration of a compound, such as a chemical compound, for example an antibiotic molecule or drug that leads to activation of gene expression. Such systems are well described in the art and are known to the skilled person.

Herein the term “bicistronic” or “bicistronically” refers to a nucleic acid molecule having or comprising two cistrons, wherein a cistron is a loci responsible for generating a protein (e.g., a gene), or in other words a unit of hereditary material (such as DNA or RNA) which encodes a protein. The expression of the genes/cistrons encoded by a bicistronic nucleic acid molecule can be referred to as bicistronic expression.

A gene expression system that may be used in the context of the invention is a system specifically designed for the production of a gene product of choice. This is normally a protein although may also be RNA, such as micro-RNA. An expression system consists of a gene, normally encoded by DNA, and the molecular machinery required to transcribe the DNA into mRNA and translate the mRNA into protein using the reagents provided. An expression system is therefore often artificial in some manner; however, certain parts of the machinery required for gene expression may be provided by the target cell.

For example, in some embodiments, inducible and/or controlled gene expression can be achieved by the use of antibiotic-controlled transcriptional activation. Antibiotic-controlled transcriptional activation is a method of inducible gene expression where transcription is reversibly turned on or off in the presence of the antibiotic or one of its derivatives (e.g. blasticidin, puromycin, doxycycline, or tetracycline).

In the context of the invention the term transcription factor (TF) relates to a protein that controls the rate of transcription of genetic information from DNA to messenger RNA, by binding to a specific DNA sequence. The function of TFs is to regulate (turn on and off) genes in order to make sure that they are expressed in the right cell at the right time and in the right amount throughout the life of the cell and the organism. Groups of TFs function in a coordinated fashion to direct cell differentiation, cell division, cell growth, and cell death throughout life; cell migration and organization (body plan) during embryonic development; and intermittently in response to signals from outside the cell, such as a hormone. TFs work alone or with other proteins in a complex, by promoting (as an activator), or blocking (as a repressor) the recruitment of RNA polymerase (the enzyme that performs the transcription of genetic information from DNA to RNA) to specific genes. A defining feature of TFs is that they contain at least one DNA-binding domain (DBD), which attaches to a specific sequence of DNA adjacent to the genes that they regulate.

Transcription factors can be used for reprogramming or directed differentiation of mammalian cells to a different cell type. Induction of a different cell type in an initial cell/staring cell can be achieved through provision of one or more TF. In the context of the present invention, the term “initial cell” relates to a cell that is used as a starting point for inducing a steroidogenic phenotype in this cell, wherein at least the TF SF1 is provided in the cell. In the context of the invention, any kind of cell, preferably a mammalian cell can be used as an initial cell. Preferably the initial cell is a human cell. A cell is the basic structural, functional, and biological unit of all known living organisms. A cell is the smallest unit of life. Cells are often called the “building blocks of life”.

Preferable initial cells of the present invention are pluripotent or multipotent mammalian cells, including stem cells. Preferably the initial cell is a mammalian, preferably a human embryonal stem cell, a human embryonal stem cell line or an induced pluripotent stem cell (iPSC). Herein the terms “embryonal” and “embryonic” may be used interchangeably. Accordingly, an embryonal stem cell may be an embryonic stem cell and vice versa. Herein, the term “pluripotent stem cell, PSCs” may in some embodiments summarize or comprise both “induced pluripotent stem cells, iPSCs” and “embryonic stem cell, ESCs”. In other words, in some embodiments the PSCs are the generic of both iPSCs and ESCs.

iPSCs are a type of pluripotent stem cell that can be generated directly from adult cells. iPSC can propagate indefinitely in cell culture, as well as give rise to every other cell type in the body or the respective mammalian organism, such as the human organism, including neurons, heart cells, pancreatic cells, and liver cells, they represent a single source of cells that could be used to replace those lost to damage or disease. The most well-known type of pluripotent stem cell is the embryonic stem cell (ESC). However, since the generation of embryonic stem cells involves manipulation of the pre-implantation stage embryo, there has been much ethical controversy surrounding their use. Further, because embryonic stem cells can only be derived from embryos, it has so far not been feasible to create patient-matched embryonic stem cell lines. Since iPSCs can be derived directly from adult tissues, they not only bypass the need for embryos, but can be made in a patient-matched manner, which means that each individual could have their own pluripotent stem cell line. These unlimited supplies of autologous cells could be used to generate transplants without the risk of immune rejection. Furthermore, iPSC and iPSC derived cells can be used in personalized drug discovery efforts and understanding the patient-specific basis of disease. This also applies to the induced steroidogenic cells of the present invention that can be derived from human patient specific iPSC. iPSCs are typically derived by introducing products of specific sets of pluripotency-associated genes, or “reprogramming factors”, into a given cell type. The original set of reprogramming factors are the transcription factors Oct4 (Pou5f1), Sox2, cMyc, and Klf4. While this combination is most conventional in producing iPSCs, each of the factors can be functionally replaced by related transcription factors, miRNAs, small molecules, or even non-related genes such as lineage specifiers. Such replacement of factors required for cellular reprogramming also applies to other cellular reprogramming efforts.

Steroidogenic enzymes are enzymes that are involved in steroidogenesis and steroid biosynthesis. They are responsible for the biosynthesis of the steroid hormones, including sex steroids (androgens, estrogens, and progestogens) and corticosteroids (glucocorticoids and mineralocorticoids), as well as neurosteroids, from cholesterol. Steroidogenic enzymes are most highly expressed in classical steroidogenic tissues, such as the testis, ovary, and adrenal cortex, but are also present in other tissues in the body. Steroidogenesis is the biological process by which steroids are generated from cholesterol and changed into other steroids. The major classes of steroid hormones, as noted above, are the Progestogen, Corticosteroids (corticoids), Androgens, and Estrogens.

Herein a steroidogenic cell is a cell that is in some embodiments capable of producing steroidogenic enzymes for steroidogenesis and steroid biosynthesis. In preferred embodiments the cell is also capable of steroidogenesis and/or steroid biosynthesis. In some embodiments the steroidogenic cell according to the invention is capable of synthesizing and/or producing one or more steroid hormones selected from the group comprising steroid hormones, sex steroids (androgens, estrogens, and progestogens), corticosteroids (glucocorticoids and mineralocorticoids), neurosteroids and cholesterol. In embodiments the genes encoding essential enzymes required for steroidogenesis and steroid biosynthesis may be present naturally inside the cell and/or may have been introduced into the cell exogenically. In a one embodiment the steroidogenic cell is produced from a pluripotent cell (initial cell) that has been differentiated into a steroidogenic cell and expresses some or all of the essential genes for steroidogenesis and steroid biosynthesis naturally.

In preferred embodiments (de novo) expression of steroidogenic enzymes and/or (de novo) production and/or secretion of steroid hormones of the present cells is responsive and/or sensitive to adrenal stimuli, such as ACTH (adrenocorticotropic hormone) stimuli. In preferred embodiments the present cells, as well as their steroid production, preferably follows in vivo the circadian rhythm and is able to react with an appropriate hormone secretion depending on the time and stimuli dose. In some embodiments the steroid production increases with the increase of the dose of the ACTH stimuli. Examples of such increased hormone secretion/production of the present cells upon ACTH dosage increase is shown in FIG. 15B/C.

The ‘responsiveness’ to adrenal stimuli, e.g., ACTH stimuli, of the present cells can comprise in embodiments a de novo and/or an increased steroid hormone secretion depending on the time and/or adrenal (ACTH) stimuli dose. In embodiments ‘responsiveness’ to adrenal stimuli refers to the cells having a basal steroid hormone secretion/production and showing an increased steroid hormone secretion upon ACTH stimulation, preferably depending on time and/or ACTH dose.

In preferred embodiments herein a ‘steroid hormone production’ of cells comprises the ‘secretion of steroid hormones’.

In some embodiments the cells according to the invention are capable of in vitro growth after partial differentiation and/or during differentiation from pluripotent cells to iSCs. In some embodiments the cells according to the invention are capable of in vitro growth after differentiation from pluripotent cells to iSCs. This proliferation capacity of the present cells enables the expansion of said cells over several cell culture passages, such that a large quantity of the present cells can be generated according to the invention (see e.g., FIGS. 7 and 8).

Without being bound by theory, it can surprisingly be observed that when the present initial cell is a pluripotent cell, such as an iPSC, ESC or ESC cell line, an improved or increased proliferation capacity of the present cells can be observed before, but particularly during and/or after differentiation of the pluripotent cells to iSCs. The superior proliferation capacity of the present cells could not be achieved when the initial cells is only a multipotent cell, such as a mesenchymal stem cell or a multipotent bone marrow cell. The use of the present method in conjunction with the initial cell being a pluripotent cell the invention achieves the surprising advantage over the prior art, that large numbers of iSCs can be generated from a small number of initial cells, e.g., patient-derived iPS cells or embryonal stem cell (lines), which is an essential prerequisite for the use of said iSCs in cell therapy.

Preferably in embodiments the ‘proliferation capability’ or ‘proliferation capacity’ of the present cells and/or the existence of cell growth-capability during or after differentiation, can be detected or determined by the detection of Ki67 expression in the cells. Ki67 positivity, especially in the nucleus, of a cell can be indicative of a positive and/or increased growth capacity and therefore expansion capacity of the cells. An example can be found in FIG. 17.

The Ki67 protein is expressed as two isoforms derived from differentially spliced mRNA variants encoded by the human MK167 gene. Both Ki-67 isoforms serve a similar function during mitosis and after nuclear envelope degradation. The Ki-67 protein is expressed in a cell cycle-dependent manner and is degraded upon cell cycle exit by the proteasome and during G1 phase of the cell cycle.

FIG. 17B shows that embodiments of the present cells can be expanded for at least 6 passages. In one example a starting population of 15,000 human pluripotent (stem) cells can be expanded at least to >1 billion cells, with a doubling time of ˜28 hours.

Steroidogenic factor-1 (SF1), a master regulator of adreno-gonadal development and function encoded by nuclear receptor subfamily 5, group A, member 1 (NR5A1). SF1 is a true effector of cell fate as it initiates a genetic program driving embryonic (mesenchymal) cells or pluripotent (stem) cells toward a steroidogenic phenotype/lineage. SF1 mutations can result in adrenal insufficiency.

Steroidogenic acute regulatory protein (STAR) mediates the transfer of cholesterol from the outer to the inner mitochondrial membrane, where it is cleaved to pregnenolone and is therefore indispensable and rate limiting for the synthesis of steroids.

The steroidogenic cells according to the present invention, may be described as protein delivery vehicles, essentially enabling the localization and expression of therapeutic gene products in particular tissues or regions of the subject's body. In some embodiments such therapeutic cells offer the potential to provide cellular therapies for diseases that are refractory to other treatments.

For each type of therapeutic cell the ultimate goal is the same: the cell should express a specific repertoire of genes, preferably exogenous nucleic acids that code for therapeutic gene products, thereby modifying cell identity to express said gene product and provide a therapeutic effect, such as restoring the production of steroid hormones.

In another aspect present invention relates to an iSC for use as a medicament wherein a plurality of iSCs are encapsulated and provided within an immunoisolating device.

Immunoisolation devices can be used to transplant cells in the treatment of numerous diseases without the need for immunosuppressive drugs. Transplanted cells are enclosed within a material, which protects the transplanted cells from the patient's immune system. At the same diffusion of oxygen, carbon dioxide, soluble nutrients, signaling molecules, and bioactive cellular secretions, including therapeutic proteins, allows for both sustained viability of transplanted cells and delivery to the host of the therapeutic molecules of interest. Three types of immunobarrier devices are available today: (1) intravascular, (2) extravascular macrocapsules, and (3) microcapsules. The technique of immunoisolation is based on the principle that allogeneic and xenogeneic cells, once segregated within a selectively permeable membrane, are protected, entirely or in part, from host immune defense, while delivering specific therapeutic proteins to the patient over an extended period of time. The vulnerability of transplanted cells to immune-mediated destruction is further reduced by the semipermeable membrane also preventing the outgrowth of the encapsulated cells into the patient's parenchyma. Accordingly, also mitotically active cells can be transplanted. Common immunoisolating devices are, although the invention is not limited to those specific devices, Enscaptra (Viacyte), PEC Direct, PEC-Encap and PEC-QT. Accordingly, the present invention also encompasses treatment of a patient by introducing a therapeutically effective number of cells into a subject, preferably by using an immunoisolation or a similar device.

The iSCs according to the invention may in some embodiments also be described as “bio pump” or “drug factory” as these cells can serve as a producing source of steroidogenic enzymes and hormones. By administering iSCs to a subject restoration of hormone production can be restored. In the sense the bio pump, that is the iSCs, allow in one embodiment for a continuous supply of adrenal gland hormones.

### FIGURES

The invention is further described by the following figures. These are not intended to limit the scope of the invention but represent preferred embodiments of aspects of the invention provided for greater illustration of the invention described herein.

FIG. 1: Overview of the steps of one embodiment of the method according to the invention or producing iSCs. The figure illustrates the natural steps in the development of steroid producing cells and the in vitro steps of one embodiment of the present invention.

FIG. 2: Overview of the steps of one embodiment of the method according to the invention for producing iSCs. The figure illustrates one embodiment of the lentiviral infection of pluripotent initial cells with expression vectors comprising either blasticidin inducible SF1 or a blasticidin inducible control gene (RFP in control vector). In one embodiment the initial cells are cultivated as 2D culture (“2D lines”), instead of classical embryo-like cell aggregates.

FIG. 3: Analysis of the gene expression in one embodiment of the steroidogenic cells according to the invention that overexpress SF1. SF1 (NR5A1) expression and StAR expression are depicted together with a scheme of the steroid production pathway (middle) and the involved enzymes (colored boxes).

FIG. 4: Analysis of the gene expression of steroidogenic enzymes and PKA signaling in one embodiment of the steroidogenic cells according to the invention. Upregulation of steroidogenic enzymes can be detected in SF1 expressing iSCs CMV promoter-driven were used to express SF1.

FIG. 5: Analysis of hormone production in one embodiment of steroidogenic cells according to the invention.

FIG. 6: Analysis of cortisol production and StAR levels in one embodiment of the cells according to the invention that were either unstimulated, br-cAMP-stimulated or stimulated with br-CAMP in pulses (S-U-S-U). Y-axis show data for day (0), 2, 4, 6 and 8 (d0, d2/D2, d4/D4, d6/D6, d8/D8).

FIG. 7: Some embodiments of the iSCs generated according to the invention can be used for high-throughput production or expansion. A timeframe and the dilutions between the passages is indicated.

FIG. 8: Some embodiments of the iSCs generated according to the invention can be used for high-throughput production or expansion, in case of 2D cultures (instead of embryo-like aggregates) said cultures can be expanded and cultured easily and space efficient.

FIG. 9: This figure compares the timeline and gene expression during the first 6 days of differentiation for some embodiments of the cells according to the invention (starting from pluripotent cells; top graph on the right of Figure; Genes are SOX2 and Oct3/4) to the timeline and gene expression in embryonic stem cells/embryo in vivo. Primitive streak (second graph from top; genes are TBXT, MIXL1), intermediate mesoderm (third graph from top; genes are OSR1, GATA3, SALL1, LHX1, and MESP1), genes during early adrenal development (bottom graph; Genes are GATA6, Dax1, WT1 and GATA4). X-axis of each graph (on the right of Figure) shows data for day 0 to day 6 (do-d6). On the Y-axis of each graph the respective gene expression is depicted.

FIG. 10: This figure depicts the gene expression (Y-axis of graphs on the right) and phenotypic appearance (pictures on the left) during the first 6 days (X-axis of graphs on the right; d0-d6) of differentiation for different embodiments of the cells according to the invention (from different pluripotent initial cells). As initial cells served either the H9-ESC cell line, the H1-ESC cell line, ACS-IPSCs or BJFF-iPSCs.

FIG. 11: The figure illustrates one embodiment of the invention. Shown is a strategy to generate human steroid-producing organoids from pluripotent stem cells. Since both the adrenal cortex and the kidney derived from the intermediate mesoderm progenitors in the primitive streak, protocols according to the invention were used to generate intermediate mesoderm from PSCs.

FIG. 12: The figure illustrates one embodiment of the invention. SF1 CRISPR-Cas9 genome editing strategy. A template with a 2A-EGFP-loxP-PGK-Puro-loxP-pA floxed by the final intron/exon and the 3′UTR regions of SF1 has been cloned and electroporated with Cas9 and designed gRNAs in the H9 hESC line. The resulting puromycin resistant clones were selected and puromycin resistance was excised using Cre recombinase. Primers FW1 and RW1 were used to confirm the correct integration of the cassette in the genomic DNA.

FIG. 13: The figure illustrates two embodiments of the invention. Two schemes of cell encapsulation devices are depicted. The first scheme (top) depicts alginate-immobilized steroid-producing cells/organoids receive solutes and stimuli through a porous membrane impregnated with alginate and oxygen through the gas chamber located in the core of the device. The device includes access ports for exogenous oxygen refueling. The second scheme (bottom) shows an illustration of the encapsulation of hisCs according to one embodiment of the invention into a medical, immunoisolating device. In this embodiment the device enables the provision of adrenal hormones by the encapsulated hiSCs to the patient (when implanted or connected to the patient) and at the same time the provision of nutrients and ACTH from the patient to the encapsulated cells. In addition, the device protects the cells from the immune system of the patient.

FIG. 14: (A) Schematic of the protocol to generate mesodermal cells. The resulting cells were either not infected with lentivirus or infected with lentivirus encoding MC2R/MRAP and/or SF1. (B) Schematic of the vector utilized for the overexpression of bicistronic MC2R/MRAP under the CMV promoter/enhancer (CMVprom/CMVenh). (C-E) Expression levels of NR5A1 (SF1; C), MC2R (D) and MRAP (E) (normalized over actin expression levels) determined by RT-qPCR in cells generated according to the protocol depicted in (A), namely control cells (NI) or cells according to the invention infected with lentivirus encoding MC2R/MRAP and/or SF1 upon treatment/stimulation with (+) ACTH or without (−) stimulation (X-axis). Y-axis depicts Fold change of respective gene expression compared to SF-1+MC2R+ACTH. (F) Cortisol and (G) Aldosterone production of cells generated according to the protocol depicted in (A), namely in control cells (NI) or cells according to the invention infected with lentivirus encoding MC2R/MRAP and/or SF1, upon treatment/stimulation with (+) ACTH or without (−) stimulation (X-axis). Y-axis depicts the concentration (of steroids secreted by cells) (F) cortisol in ng/ml, or of (G) aldosterone in pg/ml.

FIG. 15: (A) Schematic of ACTH signaling pathway in steroidogenic cells. (B-C) Cortisol (B) or aldosterone (C) secretion of the cells according to the invention after infection with the gene SF1 or the SF1+MC2R+MRAP genes upon ACTH-stimulation/administration. The production/secretion of cortisol (B) and aldosterone (C) is increasing with administration of increasing ACTH dosage. The bar plots for cortisol (B) and aldosterone (C) show the ACTH dosages of 0 nM (−) ACTH, 10 nM ACTH and 1 uM ACTH from left to right in two panels. The panel (3 bars) on the left side of each graph (B/C) shows data for cells infected with the SF1-gene, the panel on the right (3 bars) shows the data for cells with combined infection with the genes encoding SF1, MC2R and MRAP (SF1+M/M). Data for cortisol (B) is shown on the y-axis in ng/ml, the data for aldosterone (C) is shown on the y-axis in pg/ml. D) shows the cortisol excretion of the cells according to the invention upon infection with the transgenes SF1+MC2R+MRAP. Cells were in vitro either not treated for 4 days (−) (condition 1, first and third bar from the left of graph), treated for 4 days with ACTH (+) (condition 2, second and fourth bar from the left of graph) or treated for 2 days with ACTH and 2 days with ACTH withdrawal (−) (condition 3, bar on the very right of graph). Hormone measurements were performed at day 2 and day 4. The cortisol concentration is shown on the Y-axis in ng/ml.

FIG. 16: The Figure shows fluorescent microscopy pictures of the hiSCs cells according to the invention. The panels/separate pictures of the Figure show the following: The very left row of pictures shows cells infected only with the SF1-gene (see X-Axis), the second row of pictures from the left shows cells infected with the SF1-, MC2R- and MRAP-gene. The second row of pictures from the right depicts cells infected with SF1-, MC2R- and MRAP-gene and ACTH stimulation (administration of ACTH). The row of pictures on the very right side shows cells infected with SF1-, MC2R- and MRAP-gene and ACTH stimulation, but with a “control” staining without primary antibody (only secondary antibody), to ensure the specificity of MC2R antibody. The very top line of pictures shows nuclear DAPI staining of the cells. The second line from the top shows intrinsic EGFP signal of the cells upon SF1 vector infection (the vector is bicistronic with EGFP). The second line of pictures from the bottom shows MC2R-specific staining with an anti-MC2R antibody. The line of pictures at the very bottom shows a merged or overlay view of all three stainings, revealing the cells showing a double positive staining (expression) of SF1 and MC2R/MRAP and hisCs upon ACTH stimulation. This figure evidences the ACTH-responsiveness of the cells.

FIG. 17: The pictures show the results of experiments analyzing the proliferation capacity of the hiSCs cells according to the invention over multiple passages. (A) shows fluorescent microscopy pictures of the hiSCs cells according to the invention, after nuclear (DAPI) staining on the very left panel and an anti-Ki67-staining in the middle panel. Ki67-staining positive cells are indicated in the middle/central picture by white arrows. The combined staining/overlay picture of nuclear and Ki67 staining in the picture on the right side of picture (A) evidences the nuclear localization of Ki67, as indicated by the white arrows. The Ki-67 protein (also termed MKI67) is a cellular marker for proliferation, which is absent in resting cells (Go-phase of the cell cycle). B. shows a cell count over 7 passages of cultivated hiSCs cells according to the invention. The proliferation capacity is high over at least 7 passages, showing the surprisingly high and stable proliferation and expansion capacity of the herein described cells.

FIG. 18: Vector comprising bicistronically a multicloning site and an EGFP gene, which was used in the experiment shown in FIG. 17 for infection with SF1 (the empty vector is commercially available) and enabled parallel expression of EGFP and SF-1 in the infected cells.

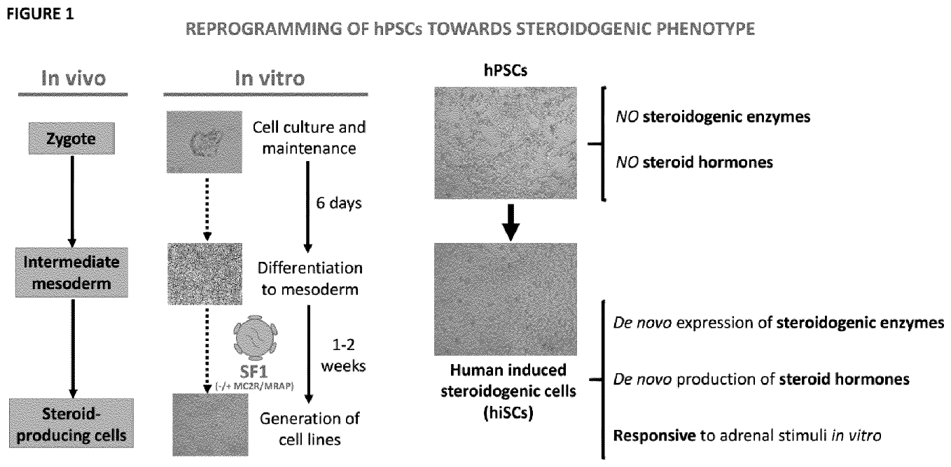

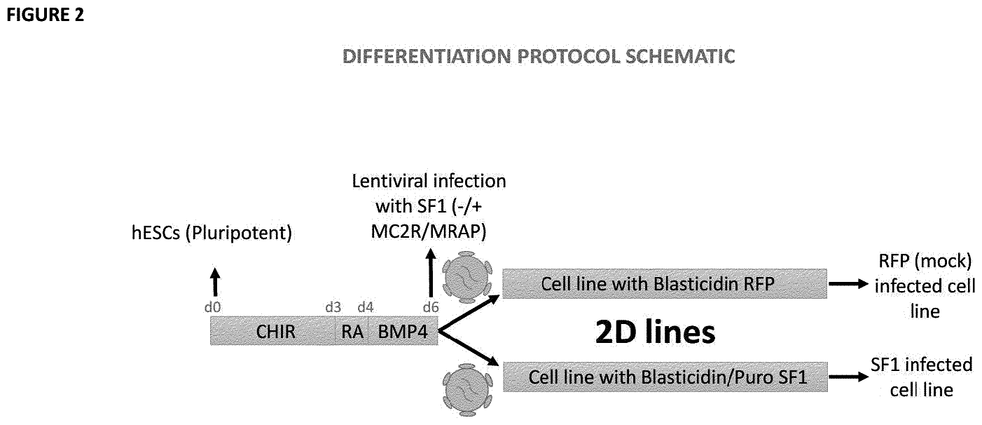

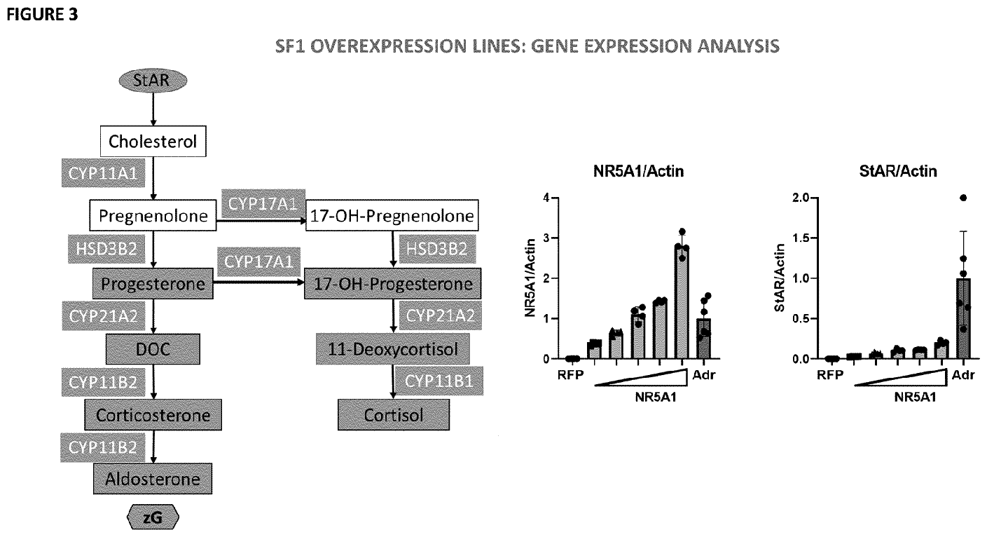

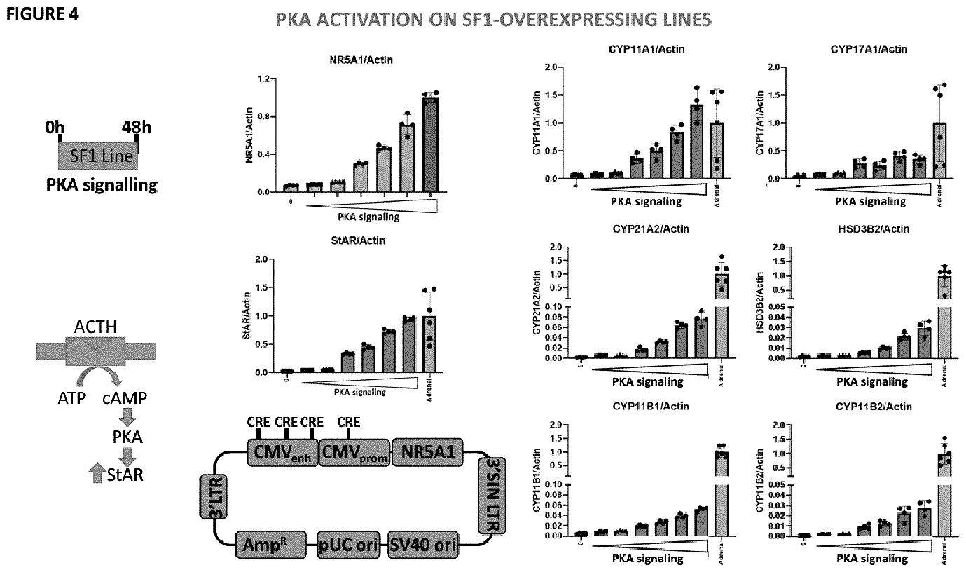

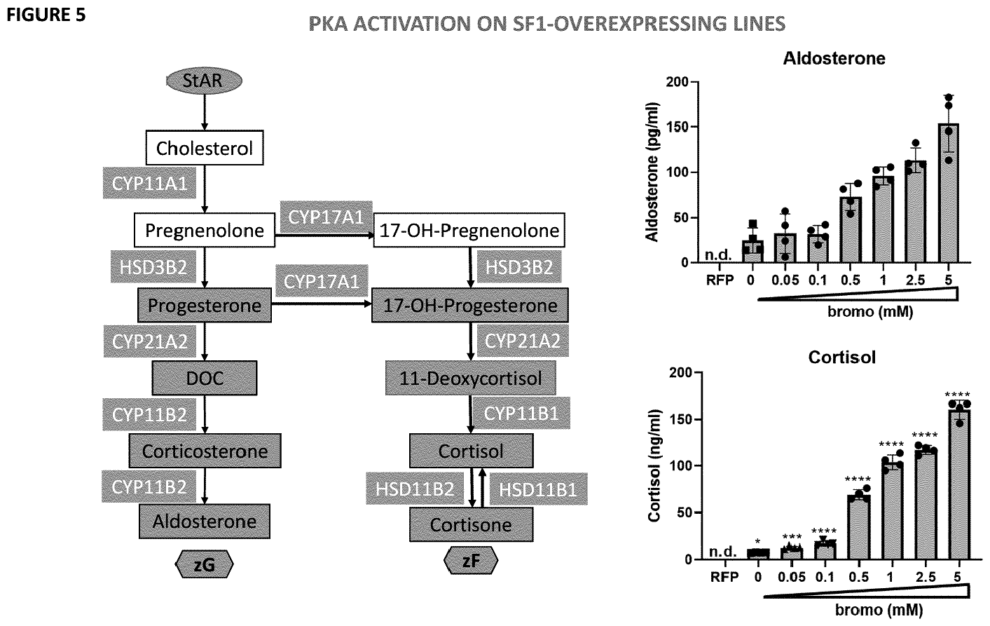

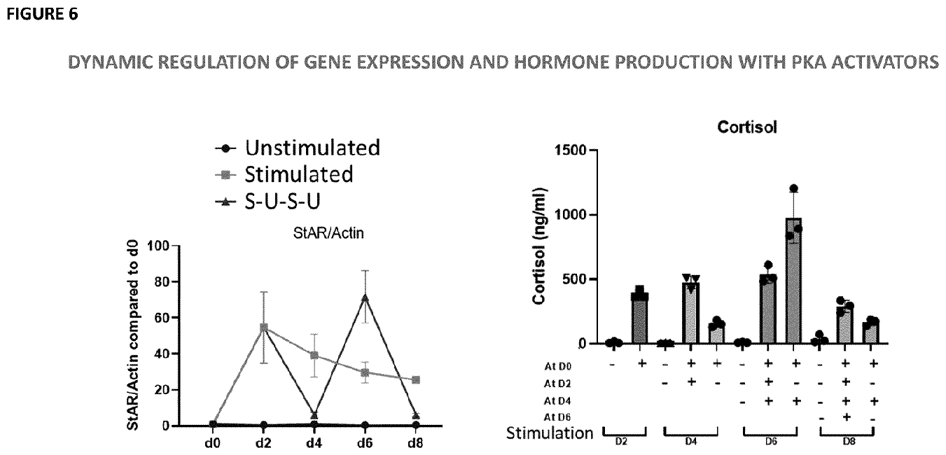

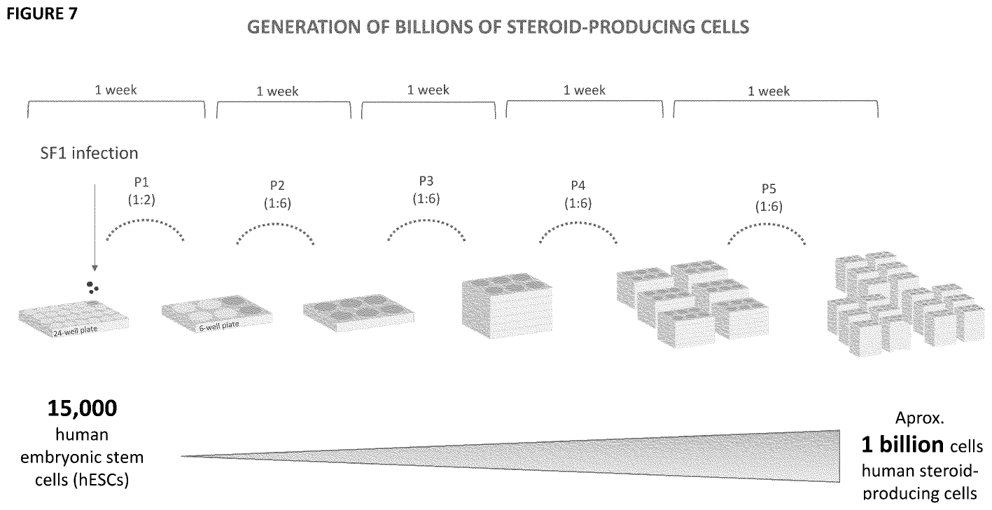

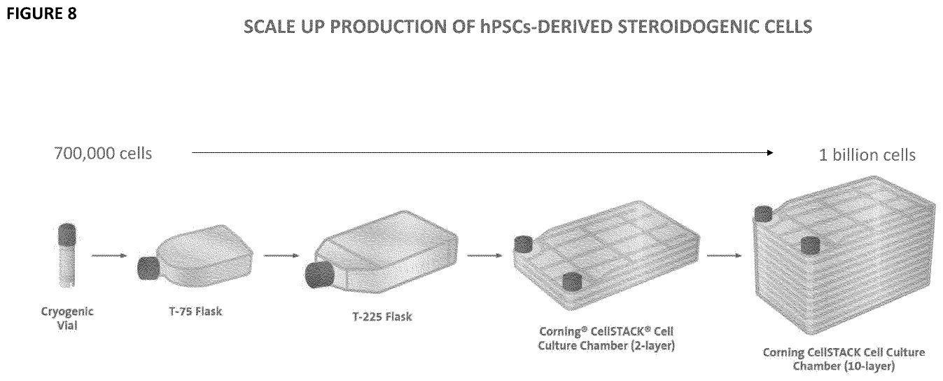

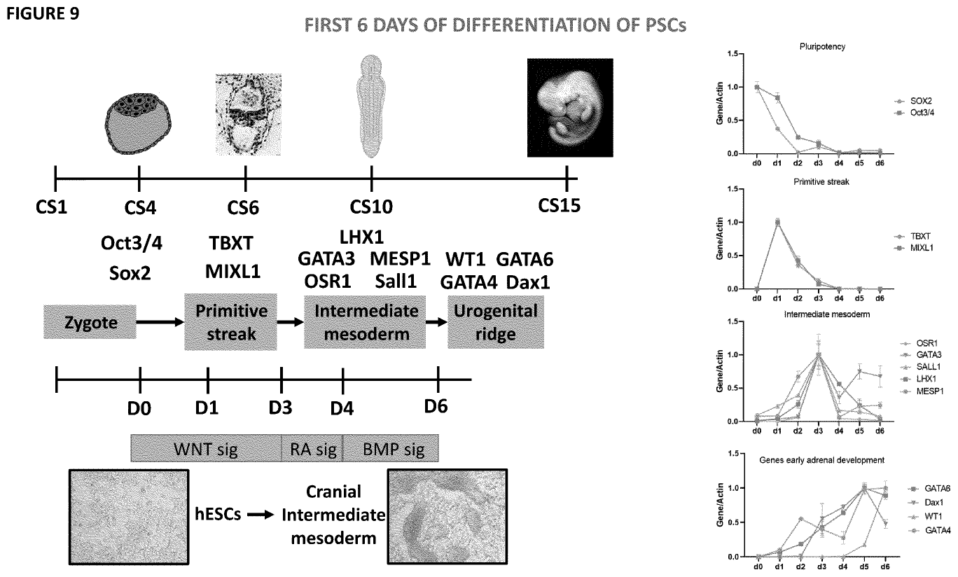

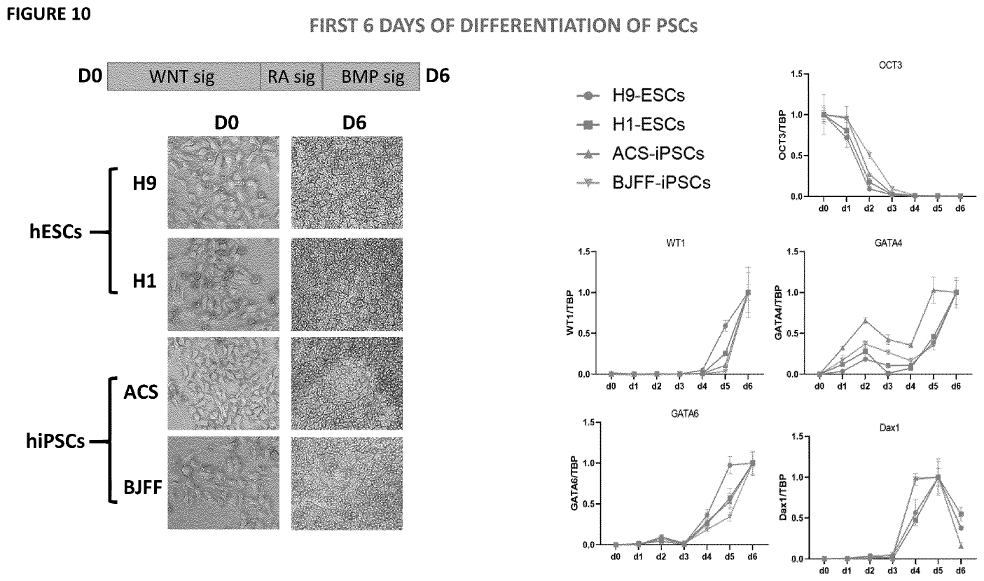

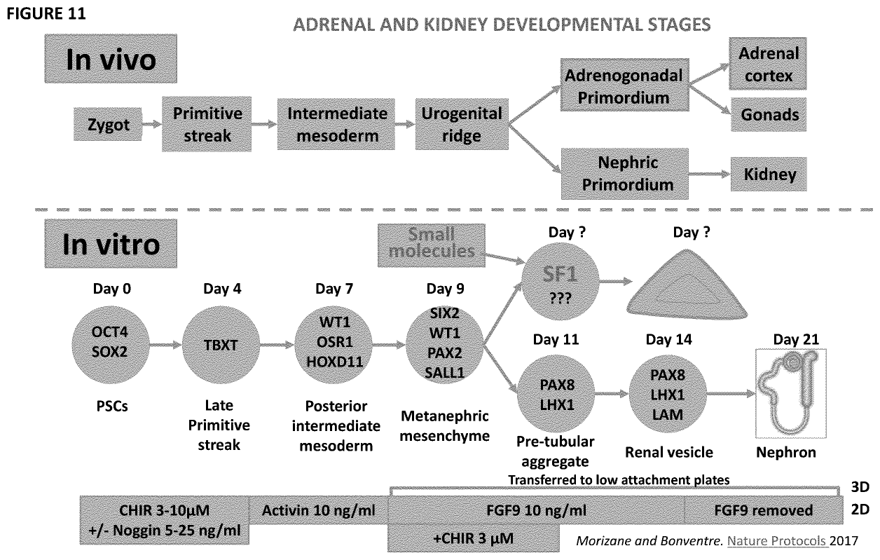

### EXAMPLES

The invention is further described by the following examples. These are not intended to limit the scope of the invention but represent preferred embodiments of aspects of the invention provided for greater illustration of the invention described herein.

### Example 1

Protocol Differentiation of PSCs into Steroid-Producing Cells

### Methods Employed in the Example

### Reagents

Accutase (StemCell Technologies, cat. no. 07920).
Advanced RPMI 1640 (Life Technologies, cat. no. 12633-020).
CHIR99021 (Tocris, cat. no. 4423)
DMEM/F12 (Life Technologies, cat. no. 11320-033).
DMSO (Tocris, 5×5 ml, cat. no. 3176)
Geltrex (LDEV-free hESC-qualified) (Life Technologies, cat. no. A1413302).
H9 hESC line (WiCell, cat. no. WA09) or H1 (WiCell, cat. no. WA01).
iPSCs, passages 22-42
Human FGF2 (Peprotech, cat. no. 100-18B)
I-GlutaMAX (Life Technologies, cat. no. 35050-061)
PBS (Life Technologies, cat. no. 10010-049)
Stem Fit Basic medium (Ajinomoto, cat. no. ASB01).
Y-27632 dihydrochloride (Tocris, cat. no. 1254)
BMP4 (Peprotech, cat. no. 120-05).
Retinoic acid 0695/50 R&D Systems.
Penicillin-Streptomycin (10,000 U/mL) (Gibco 15140122).
NR5A1 lentiviral particles (Dharmacon OHS5900-219582130, clone PLOHS_100073346

### Protocol

Maintenance of hPSCs in Feeder-Free Culture Using StemFit Basic.
All plates must be coated with 1% LDEV-free hESC-qualified Geltrex diluted in DMEM/F12.
Thaw 100.000 hPSCs per well of a 6-well plate in 1.5 ml of StemFit Basic+10 ng/ml FGF2+10 UM ROCK inhibitor Y27632.
After 3 days, change medium to 1.5 ml of StemFit Basic+10 ng/ml FGF2.
When cells reach 80% confluency, wash twice with PBS, add 0.5 ml of Accutase for 5 minutes and immediately add 0.5 ml of Stem Fit.
Count cells and plate 100.000 cells per well of a 6-well plate coated with 1% LDEV-free hESC-qualified Geltrex with 1.5 ml of StemFit Basic+10 ng/ml FGF2+10 μM ROCK inhibitor Y27632.
   Differentiation of hPSCs into Steroid-Producing Cells.
Day-3: Plate 15.000 cells per well of a 24-well plate coated with 1% LDEV-free hESC-qualified Geltrex with 0.5 ml of StemFit Basic+10 ng/ml FGF2+10 UM ROCK inhibitor Y27632.
Day 0: Change medium to Adv RPMI 1640+1% GlutaMAX+1% Penicillin-Streptomycin (Medium A)+3 UM CHIR99021.
Day 2: Change medium to Medium A+3 UM CHIR99021.
Day 3: Change medium to Medium A+10 UM Retinoic acid.
Day 4: Change medium to Medium A+10 ng/ml BMP4.
Day 5: Change medium to Medium A+10 ng/ml BMP4.
Day 6: Wash cells with PBS and incubate with 0.5 ml of Accutase for 5 min at 37° C. Infect 1 M cells with 1.5 ul of SF1 lentiviral particles (At 1×10¹²gc/ml) in suspension and plate into 2 wells of a 6-well plate coated with 1% LDEV-free hESC-qualified Geltrex in 1.5 ml of Medium A+10 ng/ml FGF2+10 UM ROCK inhibitor Y27632.
Day 8: Change medium to Medium A+10 ng/ml FGF2.
From day 8 onwards, cells can be passaged ⅓ using MediumA+10 ng/ml FGF2+10 UM ROCK inhibitor Y27632 in plates/flasks coated with 1% LDEV-free hESC-qualified Geltrex.
Cells can be frozen with MediumA+10% DMSO.

The cells have been generated in vitro and steroidogenic potential has been confirmed both by gene expression (gPCR and immunocitochemistry) and hormone profile (ELISA and Mass spectrometry).

### Results and Discussion of the Example

### Advantages of the Present Method and the Cells Generated According to Over the Prior Art

TABLE 1
Comparison of the cells according to the present invention and the cells generated according to the prior art

| | Present | | | | |
|---|---|---|---|---|---|
| Features | invention | Sonoyama | Tanaka | Miyamoto | Atwood |
| Cells used | hESCs and iPSCs | hESCs and iPSCs | MSCs (BMCs) | MSCs | MSCs or iPSCs |
| Species | Human | Human | Human (in vitro)/ | Mouse/Human | Human/Animals |
| | | | Mouse (in vivo) | | |
| Method | 3 d CHIR, 1 d RA and 2 d | 7 d of BIO (for | SF-1 | SF-1 | SF-1 and WT1 |
| | BMP4 (for mesoderm) + | mesoderm) + SF-1 | overexpression + | overexpression + | agonist/ |
| | SF-1 + MC2R + MRAP | overexpression + | cAMP | cAMP | antagonist + |
| | overexpression + ACTH | br-cAMP | | | cAMP |
| | (or PKA activators) | | | | |
| SF1 delivery | Lentivirus | Transfection | Lentivirus/adenovirus | Transfection | N/A |
| Vector | pLOC-Blasti | pCMFlag | Adeno type V | pIRES-Puro | N/A |
| NR5A1 | Human | Human | Bovine | Rat | N/A |
| Defined conditions | YES | NO, use of 10% FBS | No, use of 20% HS | NO, use of 10% FCS | NO, use of serum |
| Hormones | Progesterone, | Progesterone, | Progesterone, | Progesterone, | Progesterone, |
| produced | Corticosterone, | Corticosterone, | Corticosterone, | Glucocorticoid | Estradiol, |
| | Cortisol | Cortisol | Cortisol | Aldosterone | Testosterone |
| | Aldosterone | | Aldosterone | | |
| ACTH | Achieved after | Not assessed | Long term (3 to 10 | Mentioned in paper | N/A |
| responsiveness | overexpression of | | days), not short term. | but not shown, not | |
| | receptor/co-receptor | | | mentioned in the patent | |
| Application focus | Adrenal | Adrenal | Adrenal | Adrenal | Gonadal |

There are several advantages of the present method over the method of over Sonoyama et al. One is that the method of Sonoyama uses hESCs-iPSCs cultures in feeder layers of mitomycin C-treated mouse embryonic fibroblasts. In addition, the method of Sonoyama et al. requires embryoid body formation, while embodiments of the present invention only use 2-D culture. 2-D cultures have numerous advantages, such as, for example, easier maintenance and passaging, they facilitate an equal exposure of all cells to the culture medium and factors comprised therein, and they require less storage space. In addition, in 2-D cultured stem cells sometimes easier differentiate into the same lineage (same conditions for each cell), while cells in embryo-like aggregates sometimes tend to differentiate into different lineages, requiring subsequent dissolving of the aggregates and sorting of the cells. The method of Sonoyama et al. requires 14 days of embryoid body formation before the differentiation starts. Embodiments of the present method enable the initiation of differentiation between 2-3 days after plating cells. Sonoyama et al. further only achieve a 40-60% transfection efficiency using their protocol, while embodiments of the present method achieve through the lentiviral infection and, for example, blasticidin selection 100% of SF-1-positive cells. Embodiments of the present method require no cell sorting, while the method of Sonoyama et al. requires the sorting of TRA 1-60 cells. In addition, embodiments of the present method only use defined molecules, while Sonoyama et al. use serum. Finally, the present method can be expanded in some embodiments from 15.000 cells up to 1 billion cells, which is not possible or desired in the protocol of Sonoyama et al.

### Example 2

Generation of a hESCs SF1-Reporter Line Using CRISPR-Cas9 Knock-In Technology.

H9 hESCs were used to generate intermediate mesoderm. Gene expression profiling was performed by RT-qPCR and immunostaining to validate progression through each developmental stage (pluripotency markers: OCT4, SOX2; late primitive streak: TBXT; intermediate mesoderm markers: WT1, OSR1, HOXD11). Then, intermediate mesoderm generated from hESCs was used as a screening platform to identify small molecules and/or morphogens with the capacity to induce SF1 expression. Such factors include those 1) known to be involved in adrenocortical development or 2) known to modulate adrenal signalling pathways. Small molecule pathway modulators were: B-catenin pathway: Wnt4, Rspo1, Rspo3, HIR99021, BIO; Shh pathway: SANT1, cyclopamine, PKA pathway: Foskolin, db-CAMP, br-cAMP; FGF pathway: FGF-2, FGF-9; TGF-β patway: Activin-A, inhibin-α, SB431542; IGF pathway: IGF-1, IGF-2; Hippo pathway: Dasatinib, pazopanib and Rottlerin. Over-expression of SF1 in intermediate mesoderm cells, using viral transduction was used as a positive control for induction into the steroidogenic lineage.

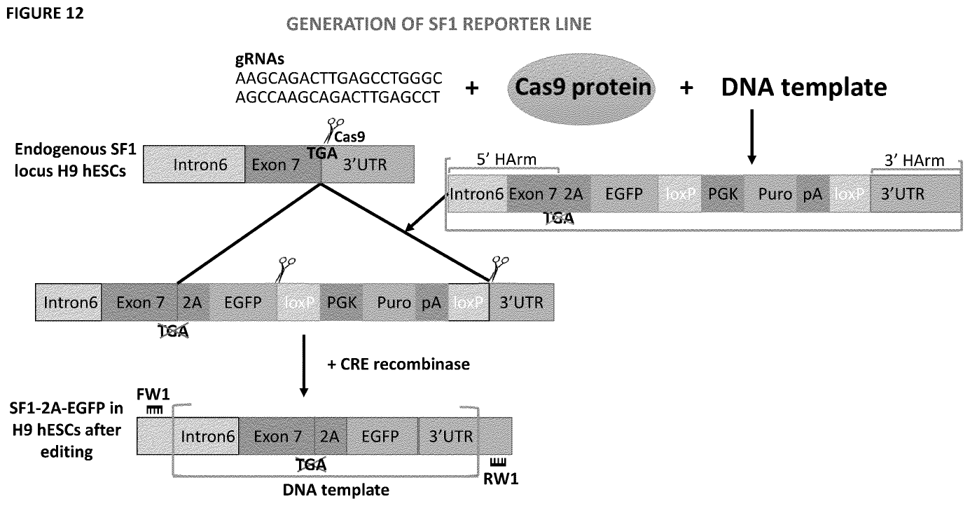

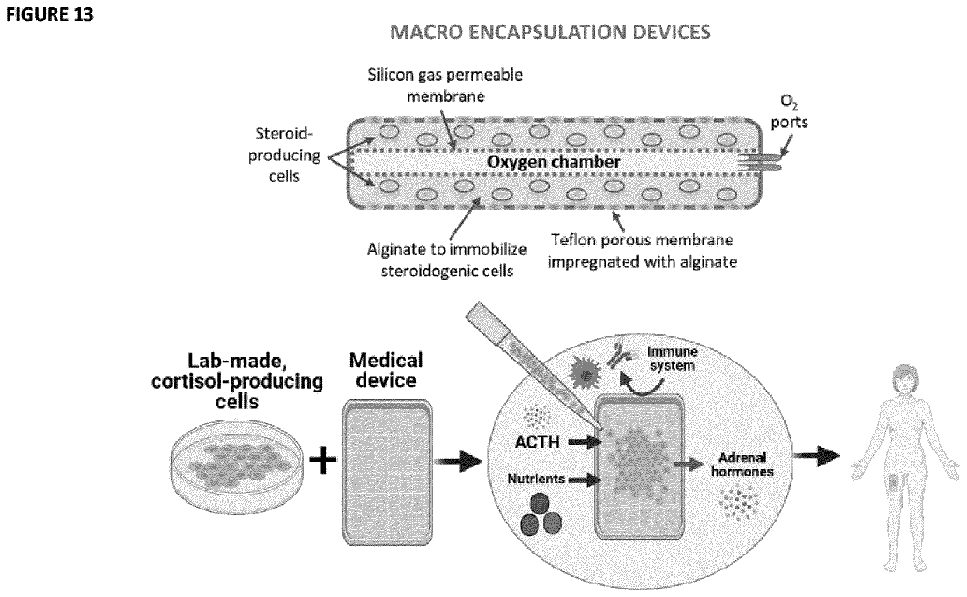

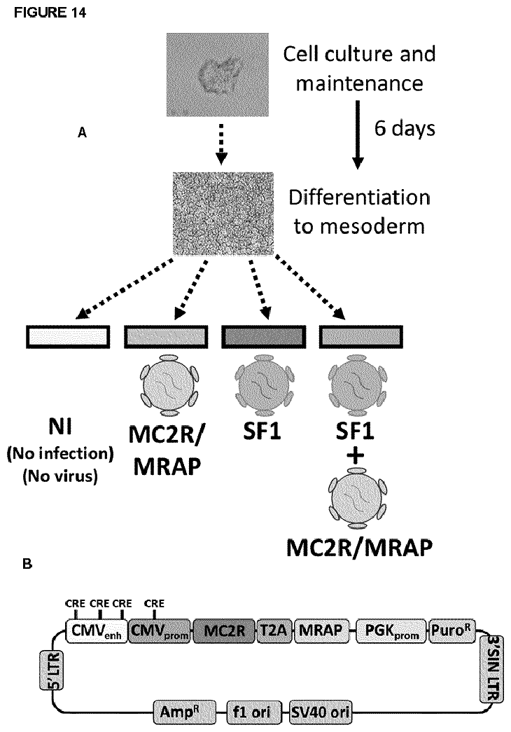

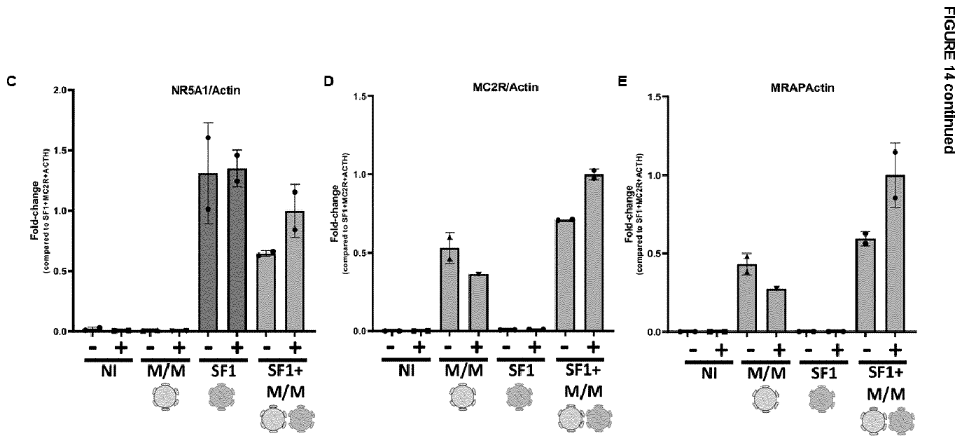

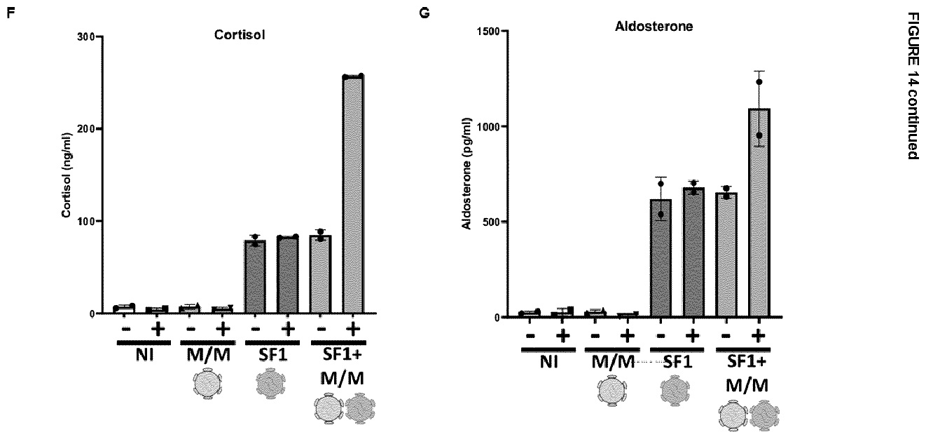

To facilitate the screening of small molecules to enhance SF1 expression, CRISPR-Cas9 genome engineering have be employed to introduce an in frame 2A-EGFP sequence into the endogenous SF1 3′UTR of H9 hESCs, followed by clonal selection to generate individual SF1-2A-EGFP-hESC reporter lines (SF1REP). A template vector with the SF1 terminal region (without the STOP codon), a loxP-flanked PKG-Puromycin cassette and the 3′-UTR homology arm was generated and optimal gRNA sequences have been selected (FIG. 12). After removal of the selection cassette using Cre recombinase, correct integration was confirmed by sequencing the endogenous SF1 locus with external primers that flank the integration site. A similar strategy has been successfully used previously to report the expression of the transcription factor MIXL1 in H9 hESCs11. SF1REP lines are treated with the compounds listed above at various concentrations, individually and in various combinations, in 96-well format. GFP expression is quantified using an EVOS FL Auto 2 Imaging System. Results from this screening strategy can define the key signalling molecules capable of inducing endogenous SF1 expression and consequently starting the steroidogenic cellular reprogramming.

Alternative to organoids generated using small molecules derived steroid-producing organoids generated through overexpression of SF1 were used.

### Biochemical Analysis of Steroid-Producing Organoids.

GFP-expressing organoids were fully characterized using RT-qPCR, immunoblot and/or immunocytochemistry for expression of a panel of steroidogenic enzymes, including: StAR, CYP11A1, CYP17A1, CYP21A2, HSD3B2, CYP11B1, CYP11B2, HSD11B1, HSD11B2 and HSD17B. The ability of organoids to produce adrenal hormones was then quantified using mass-spectrometry. The viability (Almar blue-based assay), proliferative capacity (PCNA expression, Ki-67 staining) and responsiveness to ACTH stimulation before freezing and after thawing was also assessed and confirmed.

### Use of Steroid-Producing Organoids to Model Adrenal Insufficiency.

Congenital adrenal hyperplasia (CAH), caused by mutations in steroidogenic enzymes, is the most frequent cause of primary adrenal insufficiency with a prevalence of about 1 in 18,000 worldwide. Defects in the CYP21A2 gene is the most common form of CAH. As proof of concept for the present technology, the two most prevalent mutations affecting more than 50% of patients with CAH were studied: (1) Single-nucleotide variant c.293-13A>G, which reduces CYP21A2 activity to lower than 1% 13 and (2) Aminoacidic mutation p.1172N, which results in 1-10% residual activity of CYP21A214.

To test the impact of these mutations on steroidogenesis, CRISPR-Cas9 was used to introduce these mutations into hESCs. Cells with biallelic targeting of CYP21A2 were then differentiated into steroid-producing organoids using the protocol established herein before. Subsequently, the hormonal profiles of organoids derived from hESCs harbouring CAH mutations and wild type hESCs were assessed and confirmed.

Organoids harbouring CAH mutations exhibited severe hypocortisolism. These experiments provide initial proof-of-principle evidence that gene editing strategies are useful for personalized, cell-based treatment of CAH.

### Functional In Vivo Analysis of Steroid-Producing Organoids Using Transplantation in Mouse Models of Adrenal Insufficiency.

The viability and functionality of in vitro-generated steroid-producing organoids in preclinical animal models of adrenal insufficiency in vivo was assessed.

Initial experiments assessed the engraftment potential, cell survival and functionality of steroid-producing organoids implanted under the kidney capsule or intra-adrenally. Mice were sacrificed at different time points. Immunohistochemical analysis was performed to assess implant survival, integrity and vascularization. Measurement of plasma cortisol levels (secreted from human cells but not from mouse cells) was assessed twice a week.

In a second phase, steroid-producing organoids were implanted under the kidney capsule of immunodeficient rodents with or without adrenalectomy. These experiments provide preclinical support for the potential of steroid-producing organoids as an alternative treatment for adrenal insufficiency.

### DISCUSSION

This approach represents the first attempt to generate 3-D organoid-like structures in the adrenal field. The methodology involves the novelty of using small molecules to drive the differentiation of hESCs—resulting in a viral-free, transgene-free, expandable, scalable and translatable to clinic (GMP) protocol—to generate steroidogenic cells. The approach is designed to mimic the stages of in vivo development of the adrenal gland, from intermediate mesoderm formation to regulation of key transcription factors involved in adrenal formation. The protocols used to generate adrenal-like cells can also provide insights into the processes required to generate cell types of a similar lineage, such as gonadal tissue. Finally, the ability to generate functional steroid-producing cells in 3-D organoids facilitates: i) the next generation of cell-based treatments for AI (adrenal insufficiency); ii) the modelling of adrenal specific diseases and iii) the testing of personalised interventions on cells with common mutations found in patients suffering from adrenal insufficiency (drug screening platform).

### Example 3

Small molecule screening was performed using hESCs-derived adrenal progenitors to induce endogenous SF-1 expression. Using our SF-1 reporter line, directed small-scale screening using well-established molecules involved in adrenocortical development was performed. In addition, to identify additional molecules, automatized large-scale screenings was performed. If the sensitivity of the reporter line is not sufficient to detect changes in EGFP intensity, SF-1 expression was assessed using qPCR and immunocytochemistry combined with flow cytometry.

Initial experiments assessed the engraftment potential, cell survival and functionality of steroid-producing organoids implanted under kidney capsule of adrenalectomized immunodeficient rats. Rats were sacrificed at different time points (30 days and 3 months) and immunohistochemical analysis was performed to assess implant survival, integrity and vascularization. Measurement of plasma cortisol levels (secreted from human cells but not from rat cells) was assessed and confirmed twice a week for the duration of the study. These experiments provide preclinical support for the potential of steroid-producing organoids as an alternative treatment for adrenal insufficiency.

In a second stage, encapsulation devices were filled with steroid-producing organoids and implanted under the skin of immunocompetent adrenalectomized rats. Results from this trial provide promising information on the recovery of adrenal insufficiency phenotype using this technology. As hESCs-derived steroidogenic organoids are rejected by the immune system of the host, the successful outcome of these experiments proves immune protection of encapsulated cells. Cellular therapies are emerging as an attractive alternative to drug-based treatments for hormone-released pathologies. Preclinical animal testing is a mandatory first step to move this technology from bench-to-bedside.

### Example 4

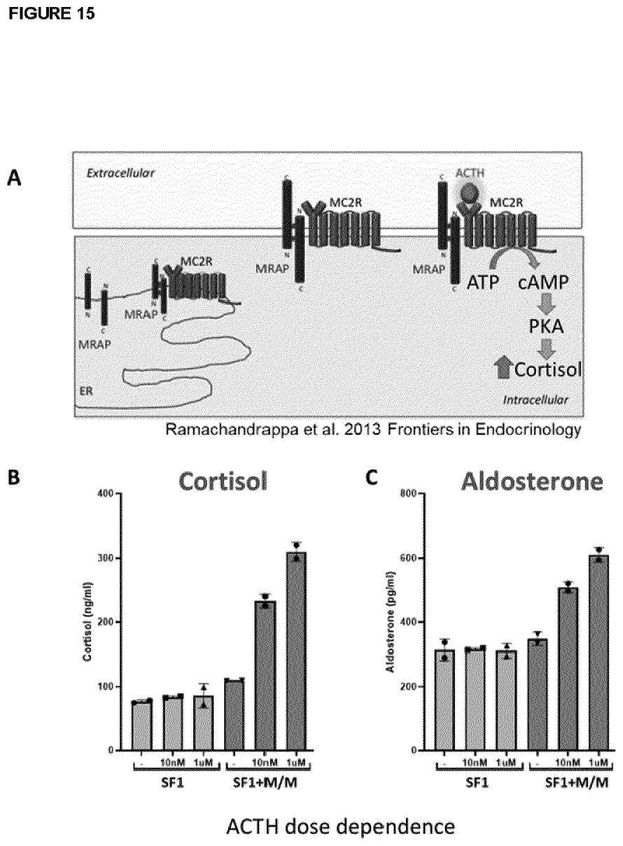

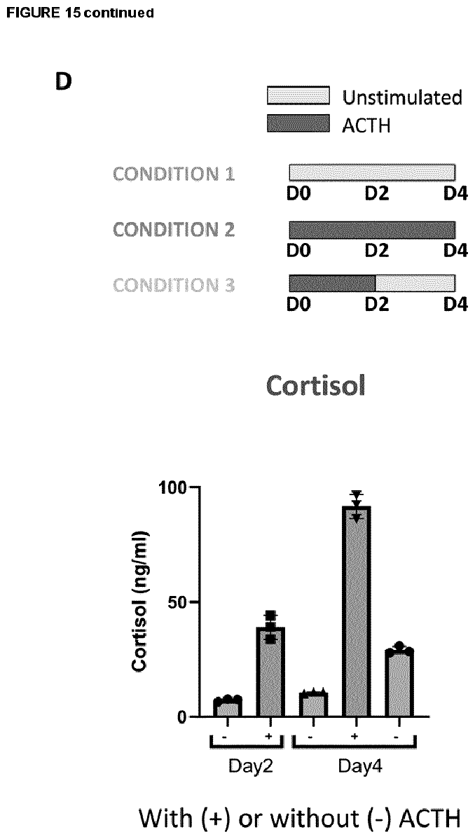

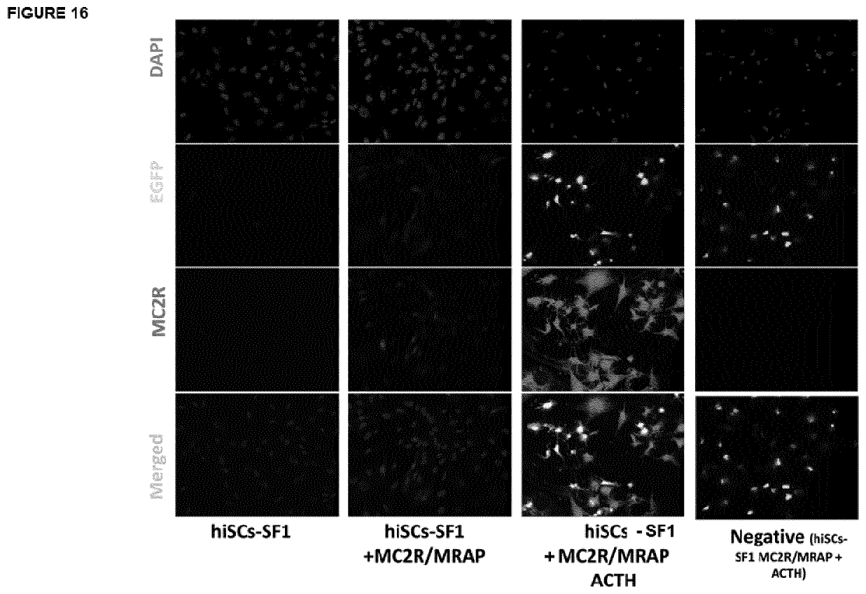

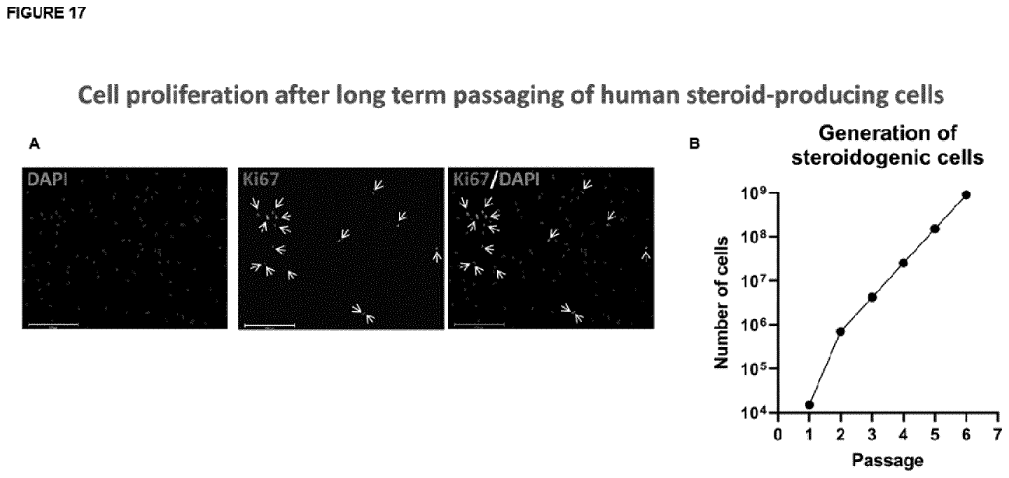

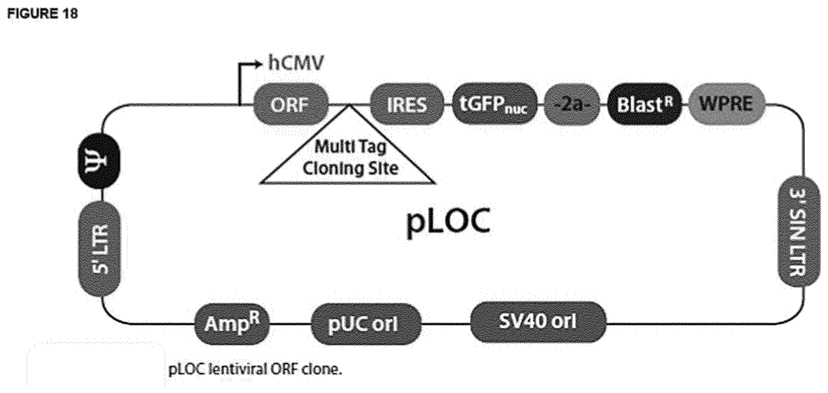

To increase the ACTH responsiveness of the present hiSCs the ACTH receptor MC2R and its co-receptor (MRAP) were cloned into a lentiviral vector, which further comprised an antibiotic resistance gene and hisCs were infected with said expression vectors. In this way, cell lines with stable MC2R and MRAP gene expression were generated, which possessed steroidogenic potential and ACTH responsiveness. The stable (over) expression of SF1, MC2R and MRAP in the cell lines was assessed by gene expression analysis. The expression of SF1 and MC2R was additionally detected using specific antibodies. The experiments showed that the ACTH responsiveness of the cells is dose and time dependent and that the cells respond to ACTH stimulation in a dynamic way. Importantly, the generated cells show ongoing growth capacity and could be amplified/expanded to obtain billions of cells, which is an essential requirement for cellular therapy. The results are shown in FIG. 15-17.

### REFERENCES

Matsuo, K., Sone, M., Honda-Kohmo, K. et al. Significance of dopamine D1 receptor signalling for steroidogenic differentiation of human induced pluripotent stem cells. Sci Rep 7, 15120 (2017). https://doi.org/10.1038/s41598-017-15485-4.
Miyamoto K, Yazawa T, Mizutani T, Imamichi Y, Kawabe S Y, Kanno M, Matsumura T, Ju Y, Umezawa A. Stem cell differentiation into steroidogenic cell lineages by NR5A family. Mol Cell Endocrinol. 2011 Apr. 10; 336 (1-2): 123-6. doi: 10.1016/j.mce.2010.11.031. Epub 2010 Dec. 4. PMID: 21134412.
Ruiz-Babot G, Balyura M, et al., Modeling Congenital Adrenal Hyperplasia and Testing Interventions for Adrenal Insufficiency Using Donor-Specific Reprogrammed Cells. Cell Rep. 2018 Jan. 30; 22 (5): 1236-1249. doi: 10.1016/j.celrep.2018.01.003. PMID: 29386111; PMCID: PMC5809617.
Sonoyama T, Sone M, Honda K, Taura D, Kojima K, Inuzuka M, Kanamoto N, Tamura N, Nakao K. Differentiation of human embryonic stem cells and human induced pluripotent stem cells into steroid-producing cells. Endocrinology. 2012 September; 153 (9): 4336-45. doi: 10.1210/en.2012-1060. Epub 2012 Jul. 9. PMID: 22778223.
Tanaka T, Aoyagi C, Mukai K, Nishimoto K, Kodama S, Yanase T. Extension of Survival in Bilaterally Adrenalectomized Mice by Implantation of SF-1/Ad4BP-Induced Steroidogenic Cells. Endocrinology. 2020 Mar. 1; 161 (3): bqaa007. doi: 10.1210/endocr/bqaa007. PMID: 31950150.
Tomoko Tanaka, Chikao Aoyagi, Kuniaki Mukai, Koshiro Nishimoto, Shohta Kodama, Toshihiko Yanase, Extension of Survival in Bilaterally Adrenalectomized Mice by Implantation of SF-1/Ad4BP-Induced Steroidogenic Cells, Endocrinology, Volume 161, Issue 3, March 2020, bqaa007, https://doi.org/10.1210/endocr/bqaa007.
Yazawa T, Kawabe S, Inaoka Y, Okada R, Mizutani T, Imamichi Y, Ju Y, Yamazaki Y, Usami Y, Kuribayashi M, Umezawa A, Miyamoto K. Differentiation of mesenchymal stem cells and embryonic stem cells into steroidogenic cells using steroidogenic factor-1 and liver receptor homolog-1. Mol Cell Endocrinol. 2011 Apr. 10; 336 (1-2): 127-32. doi: 10.1016/j.mce.2010.11.025. Epub 2010 Dec. 1. PMID: 21129436.

1. A method for producing induced steroidogenic cells (iSCs) from an initial cell, the method comprising:
providing at least one transcription factor (TF) to the initial cell,
comprising at least steroidogenic factor 1 (SF-1).

2. The method according to claim 1, wherein the initial cell is a pluripotent cell.

3. The method according to claim 1, wherein the at least one TF is expressed from one or more exogenous nucleic acid molecules within the initial cell.

4. The method according to claim 1, wherein the initial cell is provided with the at least one TF for at least 3 days.

5. The method according to claim 1, wherein the at least one transcription factor (TF) is expressed transiently and/or expression is induced in the initial cell.

6. An induced steroidogenic cell (iSC) generated by the method according to claim 1.

7. An induced steroidogenic cell (iSC), comprising an exogenous nucleic acid comprising an SF1-encoding region operably linked to a promoter or promoter/enhancer combination.

8. The induced steroidogenic cell (iSC), according to claim 7, wherein the exogenous nucleic acid comprises a viral vector and/or wherein the promoter is a constitutive or an inducible promoter.

9. The induced steroidogenic cell (iSC), according to claim 7, wherein the induced steroidogenic cell (iSC) expresses SF-1 (steroidogenic factor 1) at levels above those expressed in an induced pluripotent stem cell (iPSC).

10. The induced steroidogenic cell (iSC), according to claim 7, wherein the cell comprises one or more of the features selected from the group comprising de novo expression of steroidogenic enzymes, de novo production of steroid hormones, adrenal stimuli responsiveness and in vivo growth.

11. (canceled)

12. A method for treating a steroid deficiency, adrenal insufficiency or congenital adrenal hyperplasia (CAH),
comprising administering a therapeutically effective amount of the induced steroidogenic cell (iSC) according to claim 7 to a patient in need thereof.

13. The method according to claim 12, wherein a plurality of iSCs according to claim 5 are encapsulated within an immunoisolating device.

14. A method for producing steroidogenic hormones in an induced steroidogenic cell (iSC), the method comprising:
providing the iSC according to claim 7,
cultivating the iSC, and
isolating hormones secreted by the cultivated iSC from the cell culture.

15. A kit for producing induced steroidogenic cells (iSCs) from an initial cell according to the method of claim 1 comprising:
a vector system for providing steroidogenic factor 1 (SF1)/nuclear receptor subfamily 5, group A, member 1 (NR5A1), to the initial cell, and
reagents for detecting induced steroidogenic cells generated from an initial cell, such as
i. an antibody for the detection of one or more marker proteins, such as SF1, Steroidogenic acute regulatory protein (STAR), Cytochrome P450 Family 11 Subfamily A Member 1 (CYP11A1), CYP11A2, CYP11B1, CYP11B2, Hydroxysteroid 17-Beta Dehydrogenase (HSD3B2), HSD17B, HSD11B1, HSD11B2, CYP21A2, CYP17A1, GATA Binding Protein 4 (GATA4), GATA6, Paired Box 8 (Pax8), WT1, Dax1, Melanocortin 2 Receptor (MC2R), Melanocortin 2 Receptor Accessory Protein (MRAP) and/or SRY-Box Transcription Factor 2 (Sox2), and/or
ii. primers for detection of one or markers, such as NR5A1/SF1, STAR, CYP11A1, CYP11A2, HSD17B, HSD3B2, HSD11B1, HSD11B2, CYP21A2, CYP17A1, CYP11B1, GATA4, GATA6, Pax8, WT1, Dax1, Sox2, MC2R, MRAP or CYP11B2 by PCR.

16. The method according to claim 1, wherein the initial cell is an induced pluripotent stem cell (iPSC).

17. The method according to claim 1, wherein the at least one transcription factor (TF) is expressed from one or more viral vectors.

18. The method according to claim 1, wherein the at least one transcription factor (TF) is expressed from one or more lentiviral vectors.

19. The method according to claim 1, wherein the initial cell is provided with the at least one transcription factor (TF) for at least 7 days.

20. The induced steroidogenic cell (iSC) according to claim 7, wherein the exogenous nucleic acid comprises a lentiviral vector.
